(19) United States
(12) Patent Application Publication
(10) Pub. No.: US 2025/0040793 A1
(43) Pub. Date: Feb. 6, 2025

(54) ROBOTIC ENDOSCOPE CONFIGURATION FOR TISSUE REMOVAL

(71) Applicant: Tamar Robotics LTD, Kibbutz Yagur (IL)

(72) Inventors: Noam HASSIDOV, Moshav Bustan HaGalil (IL); Hadas ZISO, Kiryat-Tivon (IL)

(73) Assignee: Tamar Robotics LTD, Kibbutz Yagur (IL)

(21) Appl. No.: 18/714,588

(22) PCT Filed: Nov. 29, 2022
(86) PCT No.: PCT/IL2022/051274

§ 371 Date: May 30, 2024

Related U.S. Application Data
(60) Provisional application No. 63/284,668, filed on Dec. 1, 2021.
(60) Provisional application No. 63/305,342, filed on Feb. 1, 2022.

(51) Int. Cl. A61B 1/00; A61B 1/018; A61B 1/05; A61B 8/12; A61B 34/30

(52) U.S. Cl. CPC A61B 1/00154; A61B 1/00149; A61B 1/018; A61B 1/05; A61B 8/12; A61B 2034/301

(57) ABSTRACT

Systems and methods to facilitate visual and tool access within a volume reached by an endoscopic tool inserted into brain tissue. Cameras are placed in various locations distal to and/or proximal to the volume, and in some cases are repositionable. Provision of different viewing angles helps to maintain close visual monitoring of surgical progress. Retractor and scaffolding are used, in some embodiments, to open and/or maintain opening of a working volume, particularly a working volume from which material is being removed, and which may be prone to partial collapse as surrounding tissue pushes inward into the evacuated area. Working channels are reconfigurable by use of inner and outer parts, and working channels may be shaped to allow simultaneous use by a plurality of tools and/or supports.

### RELATED APPLICATIONS

This application claims the benefit of priority under 35 USC § 119(e) of U.S. Provisional Patent Application No. 63/284,668 filed Dec. 1, 2021; and U.S. Provisional Patent Application No. 63/305,342; filed Feb. 1, 2022; the contents of which are incorporated herein by reference in their entirety.

This application is co-filed with PCT Patent Application entitled “DUAL ROBOTIC ENDOSCOPE CONFIGURATION FOR TISSUE REMOVAL”, having attorney docket number 94384; its contents are incorporated herein by reference in their entirety.

### FIELD AND BACKGROUND OF THE INVENTION

The present invention, in some embodiments thereof, relates to the field of surgical devices, and more particularly, but not exclusively, to robotically controlled surgical devices.

A variety of surgeries have been demonstrated (and in some cases, are routinely performed) using robotic systems. Minimally invasive surgical procedures may have a potential benefit from appropriately designed robotic manipulators due to considerations, e.g., of size and/or flexibility. Many robotic systems operate under the close guidance of motions by a surgeon-operator.

General surgery, Urology, Gynecology, ENT surgery and Neurosurgery are a highly specialized surgical discipline with many further specialized sub-disciplines. Surgery treatment methods have been developed for the removal of tumors and other pathological material.

Endoscopic camera use is described, for example, in U.S. Pat. Nos. 9,101,268, 8,496,580, 9,101,268, 8,496,580, and/or 8,797,392.

### SUMMARY OF THE INVENTION

According to an aspect of some embodiments of the present disclosure, there is provided an endoscopic guide for brain surgery, comprising: (a) a body having a body lumen with a distal side and a proximal side; (b) a tool lumen defined within said body lumen and sized for transfer of a brain surgery tool therethrough to a distal opening on the distal side of the body lumen; and (c) at least two distal-facing imagers positioned with respective fields of view each including, from a different respective circumferential position around the distal opening, a region surrounding an axis extending longitudinally along the tool lumen out of the distal opening; wherein the distal opening subtends at least 70° of a total circumference surrounding each of the two distal-facing imagers.

According to some embodiments of the present disclosure, the at least two imagers have separate imaging optics and separate imaging detectors.

According to some embodiments of the present disclosure, the at least two imagers have separate imaging optics and share at least one imaging detector.

According to some embodiments of the present disclosure, a distance between each of the at least two imagers and the tool lumen is less than 3 mm.

According to some embodiments of the present disclosure, the endoscopic guide includes at least one additional channel within the body lumen and outside of the tool lumen.

According to some embodiments of the present disclosure, the at least one additional channel and the tool lumen and the at least two imagers rotate separately from the body lumen.

According to some embodiments of the present disclosure, the at one additional channel and the tool lumen and the at least two imagers rotate upon rotation of the body.

According to some embodiments of the present disclosure, the endoscopic guide further includes the brain surgery tool, the brain surgery tool including one or more of suction, electrical cauterization and tissue cutting tools.

According to some embodiments of the present disclosure, the brain surgery tool rotates within the tool lumen, and includes a bending region through which it bends; and extends longitudinally along a longitudinal axis of a portion of the brain surgery tool positioned distally beyond the bending region.

According to some embodiments of the present disclosure, the brain surgery tool retracts proximally along a path including a path portion along the longitudinal axis of the portion of the brain surgery tool positioned distally beyond the bending region, and a path portion passing through the bent bending region.

According to some embodiments of the present disclosure, the guide or circuitry attached thereto generates an indication when a tip of the tool extends outside of a predefined region distal to and extending from the distal side of the body lumen.

According to some embodiments of the present disclosure, the tool is operable to bend more than 90 degrees.

According to some embodiments of the present disclosure, the tool includes an imager.

According to some embodiments of the present disclosure, the tool lumen includes an imager carried at a position distally beyond the bending region.

According to some embodiments of the present disclosure, the endoscopic guide includes an imager with an imaging detector having a detector face positioned parallel to the axis extending along the tool lumen out of the distal opening.

According to some embodiments of the present disclosure, the body is rigid.

According to some embodiments of the present disclosure, the body is non-rigid.

According to some embodiments of the present disclosure, the non-rigid body bends under bending force exerted by an inserted stylet.

According to some embodiments of the present disclosure, the endoscopic guide includes a tissue support, sized and shaped to reduce ingress of surrounding tissue into a body volume within a region distal to and extending from the distal side of the body lumen.

According to some embodiments of the present disclosure, the region extends from the distal side of the body lumen with a cross-sectional profile of the body lumen.

According to some embodiments of the present disclosure, the endoscopic guide comprises an ultrasound transducer positioned on a distal portion of the tissue support, and oriented to image in a proximal direction from its position.

According to some embodiments of the present disclosure, the tissue support includes a base shaped to extend along an outer surface of the volume and a terminating end including a terminating end surface facing proximally toward the body lumen from a position located on a distal end of the tissue support.

According to some embodiments of the present disclosure, the base is sized to block tissue ingress around at least 20% of a circumference of the body volume.

According to some embodiments of the present disclosure, the base is sized to block tissue ingress around at least 40% of a circumference of the body volume.

According to some embodiments of the present disclosure, the terminating end blocks tissue ingress through an area at least 20% as large as a cross-sectional area of the body volume.

According to some embodiments of the present disclosure, the terminating end blocks tissue ingress through an area at least 40% as large as a cross-sectional area of the body volume.

According to some embodiments of the present disclosure, the terminating end faces proximally at an angle oblique to the axis extending longitudinally along the tool lumen out of the distal opening.

According to some embodiments of the present disclosure, the terminating end is shaped with curved edges that smooth its profile, so as to avoid damaging tissue as the terminating end advances through the volume to re-open a cut into the volume which has at least partially collapsed due to ingress of tissue.

According to some embodiments of the present disclosure, the tissue support is sized to insert through an auxiliary channel within the body lumen.

According to some embodiments of the present disclosure, the tissue support has a fixed shape.

According to some embodiments of the present disclosure, the tissue support is bendable.

According to some embodiments of the present disclosure, the tissue support bends to allow insertion while the brain surgery tool is inserted to the at least one tool lumen.

According to some embodiments of the present disclosure, the tissue support includes a solid wall extending over at least 60% of its surface.

According to some embodiments of the present disclosure, the tissue support includes a mesh-like wall with apertures therein.

According to some embodiments of the present disclosure, the tissue support defines at least one suction aperture facing away from the body volume.

According to some embodiments of the present disclosure, the tissue support moves axially after extending out of the body lumen.

According to some embodiments of the present disclosure, the tissue support includes at least one imager facing proximally towards the body lumen when the tissue support is extended distally out of the body lumen.

According to some embodiments of the present disclosure, the tissue support includes at least two imagers facing proximally towards the body lumen when the tissue support is extended distally out of the body lumen, the imagers being separated by a distance of at least 3 mm along a line parallel to a base of the tissue support.

According to some embodiments of the present disclosure, the tissue support is positionable to prevent contact of the brain surgery tool with sensitive tissue outside of the body volume.

According to some embodiments of the present disclosure, the endoscopic guide includes at least one narrow tissue supporter extending distally from the body lumen.

According to some embodiments of the present disclosure, the at least one narrow tissue supporter is less than 1 mm in cross-sectional extent projected towards the at least two imagers.

According to some embodiments of the present disclosure, the at least one narrow tissue supporter is in the form of a loop, with each of two sides of the loop extending distally out of the body lumen.

According to some embodiments of the present disclosure, the at least one narrow tissue supporter includes two separate wires.

According to some embodiments of the present disclosure, the at least one narrow tissue supporter rotates to move laterally relative to a longitudinal axis of the body lumen.

According to some embodiments of the present disclosure, the at least one narrow tissue supporter moves circumferentially away from the axis extending longitudinally along the tool lumen out of the distal opening upon axial advance distally from the body lumen.

According to some embodiments of the present disclosure, the at least one narrow tissue supporter has a resting position where it does not block the surgical tool.

According to some embodiments of the present disclosure, the at least one narrow tissue supporter is flexible enough to move out of the way when contacted by the surgical tool.

According to some embodiments of the present disclosure, the at least one narrow tissue supporter is re-positionable to mark a tumor or other tissue to be removed or to mark a tissue to be avoided, while the distal opening remains in place.

According to some embodiments of the present disclosure, the endoscopic guide includes a tissue support having a base extending axially along a surface of body volume distal to and extending from the distal side of the body lumen; and wherein the at least one narrow tissue supporter lies on an opposite side of the body volume from the base and extends to rest against the base.

According to some embodiments of the present disclosure, the tissue support restricts axial movement of the at least one narrow tissue supporter and thereby converts axial movement thereof into lateral and/or circumferential movement thereof.

According to some embodiments of the present disclosure, the endoscopic guide includes an ultrasound imager sized to fit through the tool lumen and image laterally.

According to some embodiments of the present disclosure, the ultrasound imager rotates within the tool lumen to a plurality of positions allowing imaging laterally to various respective directions.

According to some embodiments of the present disclosure, the endoscopic guide includes a tissue support having a base extending axially along a surface of body volume distal to and extending from the distal side of the body lumen; and wherein the ultrasound imager is sized to rest against the base.

According to some embodiments of the present disclosure, the endoscopic guide includes circuitry configured to show images from the imagers on a display.

According to some embodiments of the present disclosure, the circuitry shows the images as stereo images.

According to some embodiments of the present disclosure, the circuitry is configured to combine the images and remove at least part of the images, where view of tissue is blocked by parts of the endoscopic guide and/or tools thereof.

According to an aspect of some embodiments of the present disclosure, there is provided an endoscopic tool for brain surgery, including: (a) a body having a body lumen with a distal side and a proximal side; (b) at least one tool lumen defined within the body lumen and sized for transfer of a brain surgery tool therethrough to a distal opening on the distal side of the body lumen; (c) at least one distal-facing imager; (d) at least one tissue support extendable from the distal side of the body lumen to occupy a position that interferes with ingress of tissue into a body volume within a region extending distally from the distal side of the body lumen.

According to an aspect of some embodiments of the present disclosure, there is provided an endoscopic tool for brain surgery, including: (a) a body having a body lumen with a distal side and a proximal side; (b) at least one tool lumen defined within the body lumen and sized for transfer of a brain surgery tool therethrough to a distal opening on the distal side of the body lumen; (c) at least one distal-facing imager alongside the distal opening, and coupled to rotate along with a brain surgery tool inserted through the lumen.

(c) at least one distal-facing imager alongside the distal opening, and coupled to rotate along with a brain surgery tool inserted through said lumen.

According to an aspect of some embodiments of the present disclosure, there is provided a system for excision of a tissue portion within neural tissue, including: an introducer; an endoscope within the introducer, including two cameras oriented to image a region distal to the endoscope and the introducer; a motor-operated surgical tool; and a controller configured to operate the motor-operated surgical tool in the region distal to the endoscope and the introducer, according to commands initiated by user inputs to the controller; wherein the motorized operated surgical tool accesses the region distal to the endoscope and the introducer via a first working channel defined between the introducer and the endoscope.

According to some embodiments of the present disclosure, at least a second working channel is defined between the introducer and the endoscope.

According to some embodiments of the present disclosure, the system includes a retractor, sized to advance out of and be retracted into the second working channel, and including a tip that bends to deflect toward a central axis extending out of the introducer when advanced, and that flattens again upon being retracted again into the second working channel.

According to some embodiments of the present disclosure, the retractor includes at least two camera elements positioned on the tip, and oriented to look proximally back toward the introducer when the tip is bent.

According to some embodiments of the present disclosure, the retractor includes a groove along a side of the retractor facing toward the central axis; and including a scaffold that slidably extends from at least the second working channel into the region distal to the endoscope and the introducer; wherein a portion of the scaffold extending from the second working channel also extends along the groove of the retractor, stabilizing the scaffold.

According to an aspect of some embodiments of the present disclosure, there is provided an endoscopic surgical system including: an introducer having a proximal end and a distal end; a steerable channel, including at least one tubular element sized to pass along the introducer between the proximal and distal ends; and a robotic motor controller within an enclosure, and configured to engage with the introducer and the steerable channel within the introducer, and including actuators configured to move at least the tubular element at least along a proximal-distal axis extending through the introducer; wherein the introducer engages with the enclosure in a region extending along a corner of the enclosure, such that sides of the enclosure extend at different angles away from opposite lateral sides of the introducer.

According to some embodiments of the present disclosure, the different angles meet at an angle of 120° or less.

According to some embodiments of the present disclosure, the introducer has a longer cross-sectional axis and a shorter cross-section axis, and engages with the enclosure in a relative orientation of the longer cross-section axis which is selectable among at least two different options.

According to an aspect of some embodiments of the present disclosure, there is provided an endoscopic surgical system including: an introducer having a proximal end and a distal end, with a cross-section having a longer cross-sectional axis and a shorter cross-section axis; at least two ports extending through the introducer between the proximal and distal ends, and arranged side by side along the longer cross-sectional axis; at least one steerable channel, including at least one tubular element sized to pass along one of the ports between the proximal and distal ends; and a robotic motor controller configured to engage with the introducer and the steerable channel within the introducer, and including actuators configured to move at least the tubular element at least along a proximal-distal axis extending through the introducer.

According to some embodiments of the present disclosure, the introducer is cross-sectionally sized to pass through human nostril into a nasal sinus.

According to an aspect of some embodiments of the present disclosure, there is provided a system for excision of a tissue portion within neural tissue, including: an introducer having a distal end and a proximal end, and defining a circular inner cross-sectional area characterized by an inner diameter; an endoscope sized to fit within the introducer extending between the distal end to the proximal end, and with a cross-section including: at least one circumferential region defining across it a diameter within 0.1 mm of the inner diameter, and at least one recessed region, radially recessed from the circumferential region to define a first compound channel between the endoscope and the introducer occupying at least 10% of the inner cross-sectional area; and a distal support element, having a cross-section sized to pass fittingly along the non-circular channel in a pre-defined cross-sectional position, and long enough to pass distally along the channel to protrude from the distal end of the introducer. wherein the distal support element occupies only a portion of the first compound channel, and is shaped to define a second compound channel between itself and at least one of the endoscope and the introducer.

According to an aspect of some embodiments of the present disclosure, there is provided a method of endoscopically excavating tissue from a target tissue volume, the method including: inserting an introducer with a distal cross-section into a body to reach the target tissue volume; inserting an endoscope through the introducer to reach the target tissue volume; inserting a steerable channel through the endoscope to reach the target tissue volume; operating a tool guided by the steerable channel to excavate a first region through the target tissue volume extending distally from the introducer, the first region having a cross section sized to match the distal cross-section; advancing the introducer distally into the first region; and withdrawing the introducer proximally from a distal end of a retractor extending distally from the introducer along a first side of the first region; wherein the withdrawing exposes a scaffold defined by one or more wires extending between the distal end of the retractor and the introducer along a second side of the first region.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Instruction executing elements of the processor may comprise, for example, one or more microprocessor chips, ASICs, and/or FPGAs. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable medium to receive instructions and/or data therefrom, process them, and/or store processing results in the same or another computer readable medium. The processing performed (optionally on the data) is specified by the instructions, with the effect that the processor operates according to the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. Additionally or alternatively, sequences of logical operations (optionally logical operations corresponding to computer instructions) may be embedded in the design of an ASIC and/or in the configuration of an FPGA device. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus such as an FPGA, or other devices such as ASICs to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such inspecting objects, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1A schematically represents an introducer, according to some embodiments of the present disclosure;

FIG. 1B schematically represents a lobed endoscope within an introducer, according to some embodiments of the present disclosure;

FIGS. 2A-2D schematically represent components of endoscope, according to some embodiments of the present disclosure;

FIGS. 3A-3D schematically represent cross-sectional considerations related to features of lobed endoscope, according to some embodiments of the present disclosure;

FIGS. 4A-4B schematically represent the insertion of tools via compound working channels, according to some embodiments of the present disclosure;

FIGS. 5A-5D schematically illustrate a steerable working channel, according to some embodiments of the present disclosure;

FIGS. 6A-6D schematically illustrate rotation of steerable working channel around the main working channel axis, according to some embodiments of the present disclosure;

FIGS. 7A-7B schematically illustrate an outer channel tube operable to slide in and out from the main working channel, according to some embodiments of the present disclosure;

FIGS. 8A-8D schematically illustrate tools with different tips that could be passed via the inner working channel of steerable channel, according to some embodiments of the present disclosure;

FIGS. 9A-9E schematically illustrate operation of an endoscope together with a steerable channel within a region of body tissue, according to some embodiments of the present disclosure;

FIGS. 10A-10C schematically illustrate the use of a retractor together with a steerable channel, according to some embodiments of the present disclosure;

FIGS. 11A-11C schematically illustrate the use of a retractor together with a steerable channel, according to some embodiments of the present disclosure;

FIGS. 12A-12D show a perspective view of an L-shaped retractor, according to some embodiments of the present disclosure;

FIG. 12E show a shows a straight-base retractor, according to some embodiments of the present disclosure;

FIGS. 13A-13E schematically illustrate an L-shape retractor, according to some embodiments of the present disclosure;

FIGS. 14A-14F schematically illustrate deployment of a scaffold allowing and assist in keeping a clear workspace in the tumor, according to some embodiments of the present disclosure;

FIGS. 15A-15E schematically illustrate an introducer comprising an L-shaped retractor together with a deployable scaffold, according to some embodiments of the present disclosure;

FIGS. 16A-16B schematically illustrate endoscopic system configurations comprising introducer, endoscope, L-shaped retractor, deployable scaffold and one or more ultrasound imagers, according to some embodiments of the present disclosure;

FIGS. 17A-17B schematically illustrates an L-shaped retractor having a distal wall with hole, according to some embodiments of the present disclosure;

FIGS. 18A-18C schematically illustrate a bendable tool, according to some embodiments of the present disclosure;

FIGS. 19A-19C schematically represent a steerable bendable tool, according to some embodiments of the present disclosure;

FIGS. 20A-20C schematically represent a configuration of a modular robotic endoscope system, according to some embodiments of the present disclosure;

FIGS. 21A-21B schematically represent an expanded configuration of a modular robotic endoscope system, according to some embodiments of the present disclosure;

FIG. 21C schematically represents an alternative expanded configuration of a modular robotic endoscope system, according to some embodiments of the present disclosure;

FIGS. 22A-22B schematically represent an expanded configuration of a modular robotic endoscope system, according to some embodiments of the present disclosure;

FIGS. 23A-23B schematically represent a-port modular robotic endoscope system, according to some embodiments of the present disclosure; and

FIG. 23C schematically represents a port arrangement of a-port modular robotic endoscope system, according to some embodiments of the present disclosure.

### DESCRIPTION OF SPECIFIC EMBODIMENTS OF THE INVENTION

The present invention, in some embodiments thereof, relates to the field of surgical devices, and more particularly, but not exclusively, to robotically controlled surgical devices.

### Overview

Minimal Invasive Surgery (MIS) has been massively used in the general treatment of patient worldwide, especially the use of endoscopes to perform such MIS procedures. Many endoscopic designs have been presented and used over the years. Endoscopes both rigid and fixable could be described as comprising a long round tube with one or more cameras in the tip. Such cameras are limited by design to a Field Of View (FOV) normally spanning between 60° and 160°, up to 180°.

FOV limits could be overcome using more than one camera; for example as shown at U.S. Pat. No. 9,101,268B2, wherein a configuration of one camera looking forward and several cameras looking sideways generates a wide FOV; for example a 270° FOV. Such a wide FOV is potentially superior to the single forward-looking camera limited to 180°, although the side-viewing cameras may introduce a physical lateral gap between the endoscopic tube and the tissue viewed. This may be suitable to use in an organ such as the colon, wherein air fills the colon cavity and there is a substantial difference between the organ diameter and the endoscope diameter. For example, a colon may range from 30-60 mm in diameter, and the colonoscope may range within 10-12 mm in diameter; a 1:3 to 1:5 endoscope to cavity ratio.

If the organ's cavity is relatively smaller—for example, 15 mm in diameter for an endoscope 10 mm in diameter—a 1:1.5 endoscope to cavity ratio is set. The side-viewing cameras may then be of lowered utility, if any, as they are confined to see a smaller fraction of the overall workspace.

Again particularly in tight confines, distal tip-mounted camera may provide good vision close to the distal tip, but tissues located far from the distal tip may be viewed poorly (e.g., due to excessive foreshortening and/or self-interference).

In U.S. Pat. No. 8,496,580B2 an omnidirectional and forward-looking endoscope is described using a special lens design, apparently also suitable for use with a high ratio of cavity diameter to endoscope diameter.

In some embodiments of the present disclosure, endoscopic solutions provide a plurality of vantage points located at the distal tip of the endoscope, distinct from each other at least insofar as they provide mutual redundancy to maintain locations in the view of at least one camera during transient blockages of one or more of the remaining cameras by elements within a narrow channel extending distally from the distal tip. The narrow channel optionally has a cavity-to-endoscope diameter ratio of 1.

U.S. Pat. No. 8,797,392B2 presents an endoscope with forward looking camera, incorporating a polarizing filter mounted at the tip of the endoscope and a deployable back-looking camera that could be deployed via the endoscope's working channel. Such a solution may generate more visual information to the user by adding a new 2nd vantage point. Such design generates enhanced vision that could not be seen from an endoscope with a single vantage point. The distance between the vantage points is preferably significant (for example, distance between vantage points may range from 10 mm-60 mm. The colonoscope diameter may range, e.g., within 10-12 mm in diameter, yielding between a 1:1 and up to 1:5 endoscope-to-vantage points distance ratio). Placed closely, the two vantage points of the cameras will block each other's view. The presented solution has to pass via a colonoscope working channel and then deflect backwards about 180° to gain vision as intended. Such a backward looking solution may result in having 2 vantage points of cameras looking on the same point; therefore having more information than a plurality of cameras located at a single vantage point.

It should be understood that any of the manipulatable elements described herein (e.g., optionally including but not limited to introducer 1, endoscope 2, retractor 40, scaffold 48, middle tube 21, ultrasound imager 1600, and/or any of tools 23, 24, 25) are optionally driven by a motor or other actuator under direction of a controller. In some embodiments, the controller is configured to select, generate, and/or provide commands resulting in motion. Selection, generation and/or provision is optionally according to user inputs received, and/or according to conditions imaged and/or otherwise detected in and/or nearby the working area of tissue within which these various manipulatable elements are moving.

An aspect of some embodiments of the present disclosure relates to endoscopic devices which provide a plurality of viewing angles into a device working space. The working space is defined at a distal end of the endoscopic device, as a volume into which tools may be advanced in order to perform tasks of an endoscopic procedure.

In some embodiments, the endoscopic device is equipped with imagers (also referred to herein as “cameras”). Imagers are distinguished from each other, e.g., by using different optical elements (imaging optics), and/or by using physically separate sensing devices (detectors). Fields of view may overlap for different imagers. Center viewing angles, however, may be used to distinguish imagers. An imager's center viewing angle is the viewing angle associated with the center of images it produces. Where the imager's field of view is configurable while the imager itself remains stationary, the images used for determining the center viewing angle may be selected as those produced with the largest (angular) field of view available.

In some embodiments, cameras are positioned to monitor field-of-view locations which are highly susceptible to becoming blocked during normal operation of the endoscopic device. In particular, cameras may be positioned on a distal face of the endoscopic device, alongside a working channel of the endoscopic device out of which a relative large element such as a steerable working channel is extended in order to perform endoscopic operations such as tissue manipulation, and/or removal. A cross-section of the relatively large element may comprise, for example, a diameter of at least 30%, 40% or more of the available inner diameter of an introducer used to position the endoscopic device. Some operations performed through the steerable working channel and in need of visual monitoring may be themselves radially confined, e.g., operations performed while working in a tissue passage which is about equal to the diameter of the introducer and/or endoscope. Accordingly, operations in a large portion of the working space are obscured from any given viewpoint, particularly viewpoints of a distal-facing imager on the endoscope distal end. Significant visual blockage may also occur due to relatively minor tissue intrusions, even of only 1-2 millimeters. For example, if an outer diameter of the introducer itself (and optionally also the hollow space it is operating in) is 10 mm, and an outer diameter of a steerable working channel is about 4 mm, then there may be a gap of less than 3 mm between the two. A 1.5 mm tissue intrusion blocks half of this distance. If a tool then deviates by even just 1.5 mm into the field of view from the opposite direction, potentially nothing at all can be seen by that camera except indistinct looming forms from which little information can be understood.

In some embodiments of the present disclosure, a plurality of cameras are placed at relatively nearby locations, but in a context which in operation often results in at least one of them being obscured. In some embodiments, this context comprises adjacency to a main working channel, out of which a tool is extended during use. Optionally, cameras are placed on either side of the main working channel. In this situation, when a tool operating out of the working channel deviates axially to obscure viewing of operations by one of the cameras, the other camera remains able to capture indications of operations being performed such as where the tool is axially, and/or its orientation.

In some embodiments, the separate center viewing angles are also associated with different offsets of the imagers along a proximal-distal axis. In some embodiments, the working space is viewed from outside of it (looking proximally) on a distal side, and from outside of it (looking distally) on a proximal side. In some embodiments, one of the two above viewing positions is substituted by a viewing position outside of the working space and looking across its proximal-distal axis. Optionally, all three of these viewing position types are provided together.

In some embodiments, the endoscopic device is equipped with imagers positioned and/or positionable together to provide two different views, albeit both distal-facing or both proximal-facing, each from a different circumferential position around a proximal-distal axis of the working space. In some embodiments, the two different distal-facing or proximal-facing views each include (when not obscured) views of a same region, which may be a region surrounding the proximal-distal axis.

However, the proximal-distal axis may furthermore be an axis extending out of a working channel of the endoscopic device, also referred to herein as a “tool lumen”. The working channel is sized such that tools fitted to it, and used while extending out of it, occupy such a large portion of the field of view of either camera in a large proportion of working positions, there is little or no overlap in utility—it may be that only one camera at a time is positioned where it can convey meaningful information.

For example, seen en face from a distal side looking proximally, the tool lumen may subtend at least about 70° of are around a camera, and optionally more, e.g., at least about 80° or at least about 90°.

When the proximal-distal axis extends from a tool lumen, the circumferential positions are optionally each within about 3 mm of the tool lumen (e.g., radially within about 3 mm of the distal aperture of the tool lumen). In some embodiments, an imager is carried on a tool which protrudes distally from a working channel. The tool may be steerable to change its center viewing angle. There may be a plurality of working channels available.

In some embodiments, two imagers with center viewing angles both oriented in a shared direction along a proximal-distal axis are at least 3 mm away from each other.

In some embodiments, the imagers are positioned and/or positionable together to provide at least two of the following:
a center viewing angle looking distally through the device's working space,
a center viewing angle looking proximally through the device's working space, and
a center viewing angle looking through the device's working space in a direction substantially orthogonal to a proximal-distal axis of the device's working space.

An aspect of some embodiments of the present disclosure relates to endoscopic devices which include support elements configured to help maintain and/or restore an uncollapsed state to tissue in a working region distal to the endoscope.

In some embodiments, the support element is extended distally through the working space. The support element may comprise a distal portion which extends through a relatively large cross-section of the working space (optionally at an angle, and not necessarily within a single cross-section), compared to a more proximal portion which remains on one side of the working space. The support element thus may act to prop open a distal side of the working space, while leaving the intervening working space itself relatively clear for tool operation.

In relation to methods of removing tissue as part of a treatment, it is useful, in some embodiments, to consider a volume referred to herein as the “in-axis workspace” of an endoscopic device, or the “distal axial shadow” of a longitudinally elongated element more generally. This is a volume surrounding an axis of the element that extends longitudinally from and distal to the element; the volume having also the distal cross-section of the element, aligned to the axis in the same way as the distal cross-section. In the case of a final distal taper and/or bevel of the element, the most distal cross-section before this taper/bevel is used.

Insofar as a particular reference may be needed to sufficiently specify the cross-sectional size of an in-axis workspace for some of the present disclosure, a workspace with a cross-section more like that of the distal (but pre-tapering) outer cross-section of an introducer (if used) is more preferred. A circular cross-section may be understood if not otherwise specified; but another cross-section shape is optionally provided; for example, elliptical, oval, rectangular with rounded corners, or another shape.

The longitudinal length of the in-axis workspace is not necessarily limited in a definition sense. Practically, may be any length suitable to the overall design of the device (e.g., determinable by comparing longitudinal extents

It is noted that since it is sized to the introducer cross-section, the volume of the distal axial shadow needs to be clear (and potentially made clear by operations such as tissue removal) in order to advance the introducer further. Moreover, and particularly if tissue clearing operations into solid tissue are limited to within the distal axial shadow, then the advancing introducer may serve as a support which prevents and/or reverses tissue collapse.

Furthermore, elements extending from near the circumferential periphery of the lumen of introducer are also well-positioned to block movement of tissue, e.g., limit it to about the wall-thickness of the introducer. As a result, confining at least an initial phase of operations to remove (excavate) tissue to within the distal axial shadow has potential advantages for controlling (e.g., substantially preventing) tissue movements into new locations which may make the current position and/or shape of a tissue targeted for treatment unclear.

Optionally, for example, to assist excavations outside of the distal axial shadow, other supporting elements are brought into use: for example a distally positioned element which supports tissue on a distal side of the available working volume, a laterally positioned element which also extends distally and blocks tissue ingress into the working area of the distal axial shadow, and/or a laterally positioned element which is expandable (laterally) to positions outside the distal axial shadow. One or both of former two elements may also be useful for maintaining tissue position and/or preventing and/or reversing tissue collapse while excavations are performed within the distal axial shadow, although some amount of prior excavation may be needed to allow them to advance distally in the first place.

The expandable element in particular (also referred to herein as a scaffold or retractor scaffold) may be expanded in a controlled fashion as tissue removal proceeds so that it maintains tissue laterally beyond and in contact with it substantially in its original position. A portion of adjacent unsupported tissue may be removed, and the scaffold moved underneath it and expanded as necessary. Tissue movement that does occur during such operations, at least when movement is to immediately adjacent areas, may be incremental and substantially reversible.

It should be noted that with suitable care, the scaffold can also be used to restore partially collapsed or otherwise moving tissue to its original location, at least, so long as the original position of the introducer remains constant or otherwise well-defined (e.g., limited in its degrees of freedom, known by imaging, known by control history and/or status, and/or by other sensing). For example, in some embodiments, the scaffold is gradually rotated around a proximal-distal axis of the distal axial shadow (the introducer) as excavation proceeds. Eventually, this moves the scaffold away from a certain side of an excavated region, with the result that this region may be freed to move (e.g., partially collapse). However, when the scaffold returns to the same position (e.g., to evacuate a next layer of tissue), it will, if also returned to its original size and shape, contact the substantially the same tissue, at the same location. This has the effect of restoring its position to about the same as it was previously.

In overall effect, this potentially allows fairly strict control of the position of tissue which is being immediately addressed (e.g., tissue adjacent on either side of the scaffold which is accessible to excavation tools), even though support may not be constant, and there may be interim movements. This has potential advantages for helping to ensure that destructive treatment operations remain confined to targeted tissue, e.g., to a region of tissue defined volumetrically before tissue removal begins. Insofar as at least some uncontrolled and/or irreversible movement may occur, it is also a potential advantage to reduce the expected amount of such movements. For example, earlier excavation operations may proceed with relative rapidity due to confidence that the volume being accessed is well within the region targeted for removal. Later excavation operations may be slowed by a need to perform extra inspections and/or exert extra care to avoid damaging untargeted tissue, once uncertainty about target position is large enough. Reducing the uncertainty in target position potentially postpones the onset of such delays, and/or reduces their magnitude.

Furthermore, the scaffolding provides a potential advantage for the tissue removal itself, by holding tissue in a position which is potentially more firmly held than, e.g., if the tissue were allowed to shift and ingress freely. In effect, the scaffolding helps to pin a current working surface against the tissue beyond it, which may help operate a tissue-removal tool in a more predictable fashion (e.g., to remove a better-defined thickness of tissue during a pass, instead of simply pushing it out of the way).

Finally, the scaffolding provides potential advantages for monitoring and/or inspection of tissue removal progress. In holding the tissue up, it potentially also helps expose the tissue surface to visual inspection from one or more imager views, e.g., views provided by an optical camera and/or ultrasound device. This can help in identifying regions of bleeding, for example. It can also help otherwise identify the type of tissue which is presently superficial, e.g., to distinguish targeted tissue with a tumorous appearance from healthy tissue. The scaffolding is optionally used as a reference for measuring tissue shape. The scaffolding is optionally used to mark tissue, e.g., by ablation (e.g., RF energy administered through an electrode region exposed through an overlying layer of insulator) and/or administration of a selective or non-selective staining material. Marks (e.g., their positions, their movements, and/or their changes in depth as tissue is removed) are optionally used to help assess procedure status and/or progress.

In some embodiments, measurements indicating positions of the scaffold itself, marks made using it, and/or marks (e.g., structured light patterns) visualized in relation to a current position of the scaffold are used to help plan modifications to a procedure, e.g., to adapt tissue removal to a volume redefined based on tissue movements and/or tissue shape changes observed.

An aspect of some embodiments of the present disclosure relates to modular designs of robotically controlled endoscopic devices. In some embodiments, an introducer for a robotic arm device couples to at least one a robotic controller in order to provide robotic control to one or more arms which pass through the introducer.

The introducer optionally includes a plurality of ports sized to allow the arms to pass. Optionally, at least one of the ports is used for an endoscopic device comprising an imager.

In some embodiments, the introducer is straight and rigid. In some embodiments, each of a plurality of robotic controllers couples to a respective port of the straight and rigid introducer, each at or near an edge and/or corner of its respective enclosure. The enclosures are thereby clustered around the introducer, for example radially arranged.

In some embodiments, each of a plurality of robotic controllers couples to a respective straight and rigid introducer, each at or near an edge and/or corner of its respective enclosure. The straight and rigid introducers are optionally aligned adjacent and parallel to each other, so that the enclosures are thereby clustered around the introducer; for example radially arranged. Optionally, the introducers are positioned with more independence in orientation, i.e., converging distally to a common working area from more widely separated positions proximally.

In some embodiments, the introducers are sized and shaped to pass through a human nostril. Nostrils are commonly oblong in shape, e.g., with a minimum cross-section about 10 mm along a long axis, and about 5 mm along a short axis. There are two of them; and although divided by a septum distally, they lead to a common volume within the sinuses. In some embodiments, introducers are provided comprising a plurality of ports arranged within an oblong-cross section; e.g., two circular ports enclosed within a rectangular cross-section with rounded ends. The overall cross-section may fit within a rectangle, e.g., about 10 mm by 5 mm in dimension. Optionally, the introducers are used together in a pair, each with its own robotic controller for controlling one or more steerable channels (manipulator arms) which pass through the port(s) of the introducer. Optionally, at least one port of at least one of the introducers is occupied by an endoscope, e.g., a device providing a distally mounted camera and illumination devices. Because the introducers are independent, one of them may be withdrawn at any time, optionally to be replaced with another tool, e.g., a flexible endoscope or other device.

### Working Channels

An aspect of some embodiments of the present disclosure relates to the use of compound working channels to support dynamically reconfigurable endoscopic and/or robotic systems.

Herein, reference to a “working channel” indicates an elongated cavity used to pass and convey matter (for example tools, fluids, tissue) along an elongated body. Typically, a working channel is circumferentially continuous, and defined by a single elongated body.

A compound working channel (optionally referred to as a “temporary” working channel and/or an “auxiliary” working channel) is, more specifically, a working channel wherein the elongated body comprises two or more separate elongated bodies, each defining a separate portion of the circumference of the elongated cavity, viewed in cross-section. The two or more bodies are separable, e.g., by withdrawing one from the other by movement along their shared axis of elongation. In virtue of this, the compound working channel may be considered temporary, since it can be disestablished according to need by separation of the two bodies. Depending on details of implementation, this may leave behind a larger working channel. This may itself also be a compound working channel, or may be a “simple” working channel, that is, a working channel of the typical circumferentially continuous type.

Before separation, the two or more bodies may be held together to form the compound working channel, for example, because one contains the other, and/or because both are contained by a constraining element such as a pipe that holds them both against each other. Additionally or alternatively, the two or more bodies may be held together by interlocking shapes, attractive forces such as magnets, and/or in another way.

In some embodiments of the present disclosure, proper functioning of some elements inserted separately on a proximal side of an introducer relies on them ending up positioned in well-specified arrangements (i.e., cross-sectional arrangements) at the distal end. Other elements may also be provided optionally on an ad hoc basis. At the same distal end, there is also, in some embodiments, a need to rearrange the presence of elements, and/or their ordering along a proximal-distal axis.

Nesting compound working channels inside other working channels (of either type) provides a potential advantage by supporting a blend of well-structured and ad hoc allocation of limited cross-sectional space. Furthermore, being able to break down and reassemble compound working channels helps promote rearrangements of the position of elements along the distal-proximal axis.

More particularly, in some embodiments, one or more compound working channels is defined between, e.g., two elongate elements. The compound working channel is sized in cross-section to fittingly accommodate a similarly elongated portion of at least one third element, while also maintaining a space which is outside the contour of the third element; that is, neither part of the solid material of the third element, nor part of a lumen which the third element entirely encloses. Because it is fittingly accommodated, the third element is held in a predetermined cross-sectional position. Furthermore, at least a portion of the outside cross-sectional space may itself be re-defined as the lumen of a compound working channel now defined in part by outer contours of the third element. This lumen is optionally available for occupation by an additional fourth or more elements. The additional elements are optionally also fitted into their space; with or without defining yet another compound working channel. Optionally, the additional elements are loose and not fitted into their channel, i.e., their cross-sectional position is not fully controlled by contacts with their channel.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Endoscope, Introducer and Three Working Channels

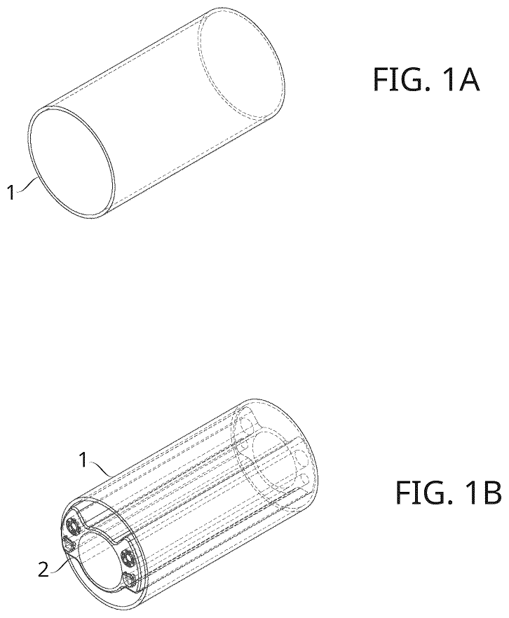

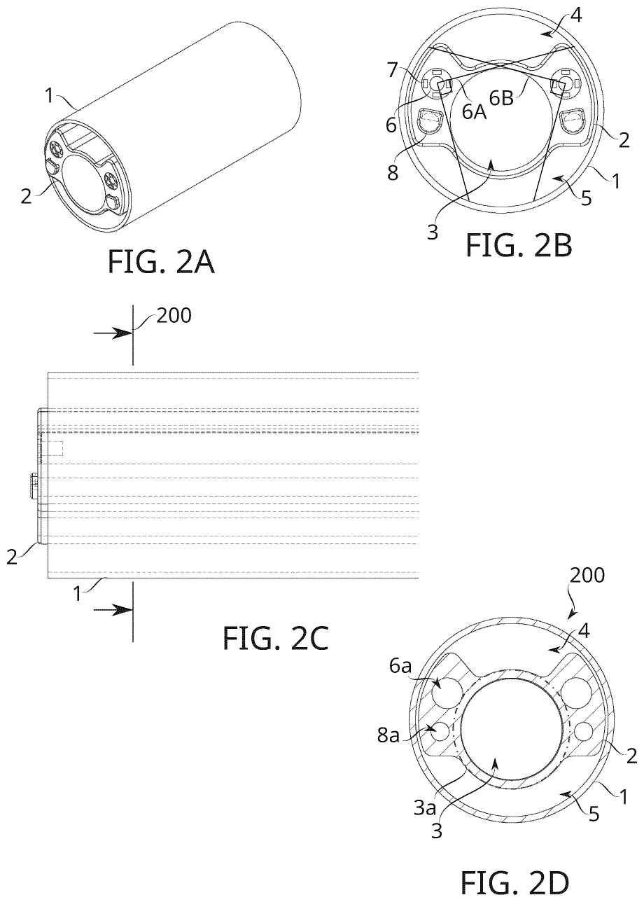

Reference is now made to FIG. 1A, which schematically represents an introducer 1, according to some embodiments of the present disclosure. Reference is also made to FIG. 1B, which schematically represents a lobed endoscope 2 within an introducer 1, according to some embodiments of the present disclosure. Broken lines indicated hidden contours.

In some embodiments, introducer 1 is a tubular body, sized and otherwise configured for insertion into body tissue (for example, brain tissue, in the context of a brain surgery), e.g., using a standard cone dilator (not shown here). Once introducer 1 is placed firmly within the tissue, the dilator is removed and retracted proximally from the introducer.

Into introducer 1, endoscope 2 is inserted. In some embodiments of the present disclosure, endoscope 2 comprises a lobed cross-section. Herein, lobed cross-sections of endoscopes comprise shapes that comprise a plurality of outer surfaces arranged to follow an inner circumference of a same radius circle (or other shape, as next explained), separated by a plurality of lower-radius sections.

Accordingly, upon insertion of the lobed endoscope into to an introducer having an inner radius matched to the radius of the outer surfaces, there is also defined between endoscope and introducer a plurality of channels, e.g., channels which may used for purposes such as the introduction of tools and/or fluids, and/or removal of material such as fluids and/or surgically dissected material.

The inner radius of the introducer 1 and the radius defining the outer surfaces of endoscope 2 “match” within some suitable tolerance, for example about 0.1 mm, the endoscope 2 being smaller. This is preferably a tolerance suitable to allow sliding motion of the endoscope device relative to its introducer, e.g., given suitable lubrication. For example the endoscope 2 is moveable along the main axis of introducer 1 (i.e., move out and in, forward and backwards, and/or distally and proximally). In some embodiments, the endoscope 2 is rotatable around the main axis of the introducer; e.g., clockwise or counterclockwise (i.e., rotate left or right and/or turn left or right).

The portion of the cross-section of the introducer 1 left open for use as a channel is, for example at least 5%, at least 10%, at least 20%, or more, e.g., in a range between about 5% and 50%.

In some embodiments, introducer 1 has, for example, an outer diameter of about 10 mm. Wall thickness may be, for example, about 0.2 mm. Optionally, introducer 1 is about 100 shorter than endoscope 2. In some embodiments, outer diameter of introducer 1 ranges between about 4-30 mm. Wall thickness ranges, for example, between about 0.1-3.5 mm. The length of introducer 1 is preferably shorter than the endoscope, for example by a distance within a range between 20-200 mm. The material of introducer 1 optionally comprises stainless steel, another metal (with preference for bio-compatible metals), polymer, and/or a single or composite composition of these or other materials. The materials may be selected and shaped to define and preserve a rigid shape which resists bending under forces experienced during use, e.g., as applied by tissue or other parts of the system. Introducer 1 is optionally (and preferably) straight.

Use of a rigid introducer (and potentially more particularly a straight and rigid introducer) may be considered a crucial requirement for certain surgical situations in which a treatment target is small, is itself sensitive to unintended damage, and/or is closely associated with tissue which it is important to avoid damaging. This includes, but is not limited to, certain neurosurgical scenarios; for example in the removal of tumors (e.g., pituitary gland tumors) and/or hemorrhagic material.

Being stiff provides potential advantages for stabilization of the introducer. Stabilization allows stable, predictable, and/or reproducible positioning of tools introduced through the introducer, including preferably a constant position maintained even in response to forces exerted through such tools externally, e.g., while interacting with tissue during a procedure.

Furthermore, tissue may move because of a procedure (e.g., during removal of nearby tissue). Accordingly, in some embodiments of the present disclosure, elements are provided which assist in mechanically stabilizing the endoscopically accessed field against collapse or other movements which may obscure or confuse the location of the treatment target. It is a potential advantage to perform such stabilizing manipulations from a platform which is itself dependably stable, so that they do not introduce positioning error, or potentially even create damage.

A straight-shaped introducer has potential advantages in particular because it is compatible with other rigid and straight-shaped tooling, including straight, rigid portions of tooling which is in part (e.g., distally) flexible. A straight access way to a target may be less resistant to longitudinal movements of elements therealong, which can be important, e.g., when treatment requires fine movements. It may be easier to determine the positioning of a straight introducer than a curved one, since its pathway of advance is straight, rather than offset. Using a curved introducer may introduce bending forces upon tools passed therealong which affects their maneuvering characteristics.

Nonetheless, introducer 1 optionally comprises a fixed-shape bend, e.g., to facilitate entry into curved body lumens, and/or to facilitate positioning of hardware elements outside the body lumen. There may be curved elements that attached to a straight section of introducer 1 on a proximal side after straight and/or rigid elements have passed distally into it. If introducer 1 has only one radius of curvature along its length, it may even be compatible with specially formed rigid tools shaped to match. Otherwise such an introducer 1 may be useful only with inserted elements which are at least somewhat flexible. The shape of introducer 1 is optionally adjustable, although if so, preferably there are provided arrangements to stiffen it after adjustment, for example, screws, spacers, internal tensioning members, or another arrangement.

Finally, non-rigid and/or non-straight embodiments of introducer 1 may be provided in some embodiments of the present disclosure. It is noted, for example, that a variably curving pathway to a target may in some cases be unavoidable, and/or (e.g. because of what it avoids) worth potential degradation of the performance of some features of the present disclosure which rigidity and/or a straight shape help promote.

In some embodiments, endoscope 2 has an outer diameter of about 9.4 mm. The gap between the inner radius of introducer 1 and outer radius of endoscope 2 may be about 0.1 mm, e.g., a diameter difference of about 0.2 mm. Length, for example, may be about 300 mm.

In some embodiments, largest outer diameter of endoscope 2 is in a range between about 4-30 mm. In a concentric (mutually centered) arrangement, the gap between endoscope 2 ranges, for example, between about 0.05-1.0 mm. In some embodiments, the gap is small enough (e.g., together with a layer of lubrication) to provide sealing, e.g., to resist the passage of moderately pressurized fluids between channels. In some embodiments, the seal is at least sufficient to resist the flow of a fluid having the viscosity of saline when a pressure differential between adjacent lobe-defined channels is 50 mm Hg or less, 75 mm Hg or less, or 150 mm Hg or less.

In some embodiments, the length of endoscope 2 is in a range between 50-750 mm. The material of the endoscope may be stainless steel, any composite of polymer or other material designed to keep shape and not bend under forces applied by the tissue or other parts of the system.

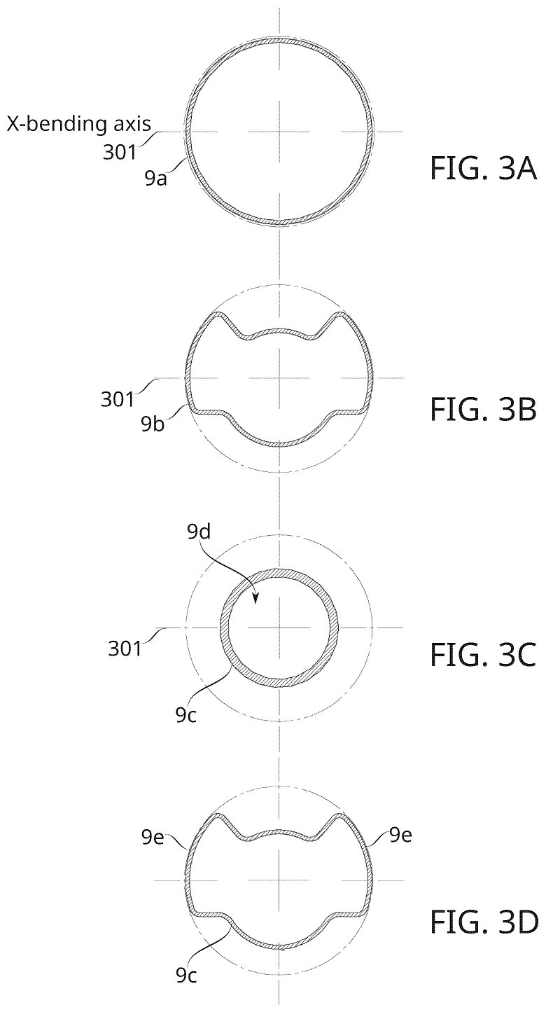

Reference is now made to FIGS. 2A-2D, which schematically represent components of endoscope 2, according to some embodiments of the present disclosure.

FIG. 2A shows a general view of the endoscope 2 located within the introducer 1.

FIG. 2B shows a front (distal-side) view. In some embodiments, a front face of endoscope 2 houses a lens 6, light sources 7 and a washing nozzle 8 configured to spray water on the lens 6 and light sources 7 to clean them from dirt. Optionally, washing nozzle 8 is used as an irrigation source to supply water into the body's cavity. Also shown in FIG. 2B is an angle marking 6A, which illustrates that main working channel 3 subtends about 90° of the circumference of lens 6. Since lens 6 is radially offset from the center of introducer 1, a larger fraction of the inner wall of introducer 1 is subtended beyond this angle. This is true of both lenses 6. The actual fields of view provided by lenses 6 are generally not a full 180°, but the conditions on the distal profile itself help illustrate the problem for less peripheral directions. Increased distance distally reduces the angular size of a large tool occupying working channel 3, but the angular size of the surrounding lumenal wall also reduces, so that blockage remains a problem.

A device operator (user) can view the workspace in looking distally from endoscope 2 via, e.g., an imager chip on a tip camera (not shown) located at a focal plane the lens 6, and/or via an external separate camera (not shown). The light sources 7 may comprise, for example, light emitting diodes LEDs located next to the front face of endoscope 2, and/or an external light emitter with light conveyed to the tip of the endoscope via a light pipe, e.g., bundles of optical fibers.

### Lower and Upper Auxiliary Temporary Working Channel

In some embodiments, a compound working channel 4 is formed between the introducer 1 and the endoscope 2 temporarily, in an upper cross-sectional area left free by the lobe-shaped cross-section of endoscope 2. In some embodiments, a compound working channel 5 is formed between the introducer 1 and the endoscope 2 temporarily, in a lower cross-sectional area left free by the lobe-shaped cross-section of endoscope 2. Optionally, one or both of compound working channels 4, 5 is used to introduce endoscopic tools. The tools may be standard/off-the-self. Optionally, the tools are specially configured for use with the system, e.g., provided with shapes fitted to the limits of the compound working channel 4, 5.

In some embodiments, compound working channel 4 has a width of about 5 mm and height of about 2.2 mm. Compound working channel 5 may have a (straight-measured) width of about 7 mm and (radial) height of about 2.2 mm.

More generally, workings channels 4, 5 are provided with a widths between about 1-25 mm (e.g., as measured in a direction extending tangentially or circumferentially) and/or a heights of about 0.5-10 mm (e.g., as measured in a direction extending radially).

FIG. 2C shows a side view of endoscope 2 within introducer 1, and indites a cross section 200. FIG. 2D indicates internal structure of the endoscope 2 at cross-section 200 (i.e., at a position proximal to the distal face view of FIG. 2B). In the example shown, endoscope 2 defines two channels 6a sized for use with optics (e.g., imaging electronics and cabling), two channels 8a operable as washing/irrigation channels to convey water to nozzle 8, and main working channel 3. Main working channel 3 has an internal diameter of, for example, about 5 mm. In some embodiments, main working channel 3 has an internal diameter within a range between about 1-25 mm. In some embodiments, main working channel 3 spans the entire longitudinal extent of endoscope 2. It may be straight (without any curves), enabling the insertion of stiff straight pipes or other tools into and through it.

### Structural Design of Endoscopes Outer Shell vs Inner Working Channel (Multi Lobe Design)

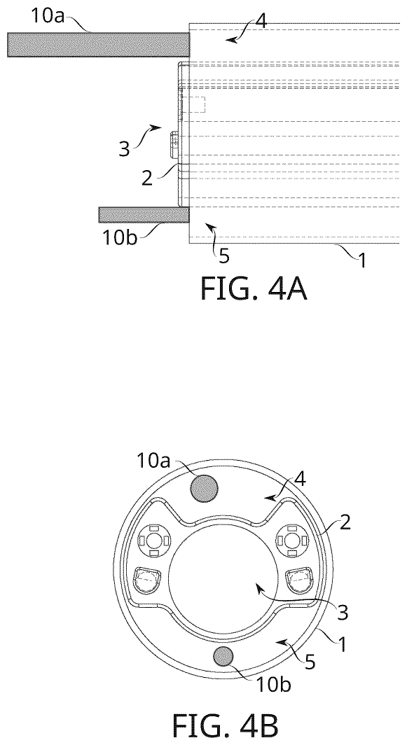

Reference is now made to FIGS. 3A-3D, which schematically represent cross-sectional considerations related to features of lobed endoscope 2, according to some embodiments of the present disclosure.

FIGS. 3A-3D show cross-sections representative the outlines of an endoscope's structural support.

FIG. 3A represents the cross-section of a standard rigid endoscope, wherein outer tube cross section 9a defines a circular wall with uniform thickness. In this example, a 9 mm outer diameter of a stainless steel structure with a wall thickness of 0.25 mm, may be supposed, similar in size to endoscopes in medical use. The “perfect pipe” shape provides the main structural element of the endoscope. The internal cavity of the pipe is used to convey fiber optics, lenses and other optic and electronic elements, optionally divided among a plurality of compartments, all entirely defined with a space defined by the outer tube.

FIG. 3B shows a lobed cross section 9b, e.g., corresponding to a cross-section of the endoscope 2 described, e.g., in relation to FIGS. 2A-2D. The non-circular cross-section presents potential challenges for design and/or manufacture while remaining compatible with other device features and/or requirements. Manufacturing of such a long non-circular part may be challenging, at least for certain preferred materials such as stainless steel.

Once manufactured, a part with a non-circular cross-section will potentially have weakened structural stiffness, compared to a circular-shaped cross-section of the same maximum diameter and wall thickness. For example, non-circular profile 9b would generally require a wall thickness of higher than 0.25 mm to gain the same bending resistance around X-bending axis 301 as pipe 9a.

In turn, the higher wall thickness reduces space available for non-structural elements significant or critical to operation of the endoscope. These typically include, for example, conduits conveying fiber optics, electronic connections, and/or fluid.

FIG. 3C shows a single pipe 9c representing a cross-section of a main working channel enabling the passage of tools via its central cavity 9d. Optionally, pipe 9c provides structural properties. For example, such a conduit with an internal diameter of 5 mm and wall thickness of 0.40 mm potentially gains the same bending resistance around X axis 301 as (thinner and wider) pipe 9a. In some embodiments, this principle is applied to the design of a lobed cross-section endoscope, for example, as shown in FIG. 3D.

FIG. 3D illustrates a compositely constructed endoscope cross section comprising a central pipe 9c, with side shells 9e and 9f attached externally. Attachment may be, for example, by welding.

Insofar as pipe 9c is relied on for stiffness, the composite cross section construction can made space efficient by reducing outer wall thickness. There may be some reliance on stiffening provided by the side-shells, so that pipe 9c need not take up all of the design requirement for stiffness alone. Accordingly, For example, the side shells 9e, 9f are optionally provided with a wall thickness of about 0.15 mm, and the internal pipe a wall thickness of about 0.20 mm.

Furthermore, the side shells can, for example, be readily formed by bending sheet stock into shape before welding, potentially promoting case in manufacturing.

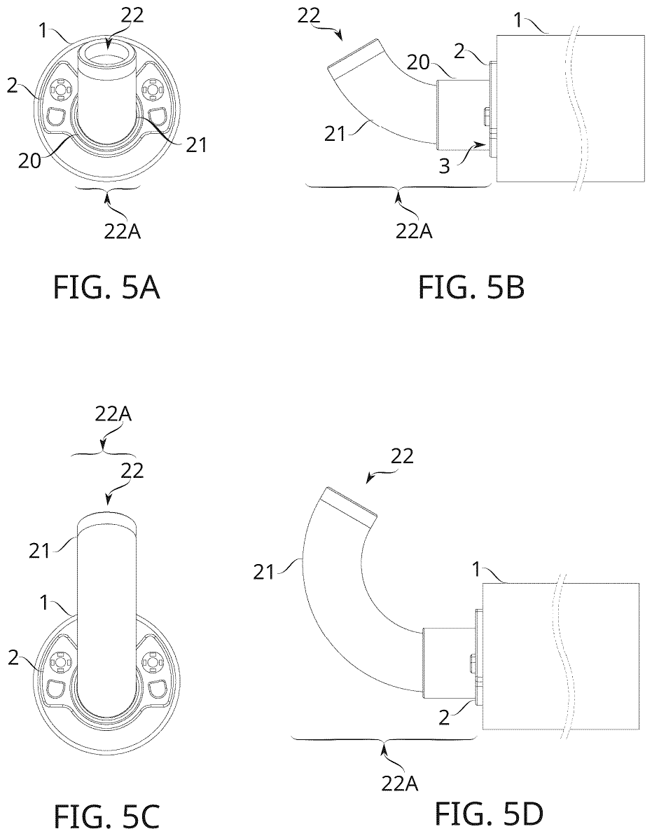

Reference is now made to FIGS. 4A-4B, which schematically represent the insertion of tools 10a, 10b via compound working channels 4, 5, according to some embodiments of the present disclosure.

FIG. 4A shows a side view, and FIG. 4B shows a front view of the endoscope 2 and introducer 1.

Tool 10b is passed within the lower compound working channel 5. Another tool 10a is passed within the upper compound working channel 4. The endoscope's main working channel 3 may be used to pass other tools if needed; for example, an ultrasound probe, therapeutic tools, and/or drugs. Optionally, the endoscope 2 is removed completely from the introducer 1 and introducer 1 used as a single very big working channel to introduce tools (not shown).

### Movements of the Endoscope's Steerable Working Channel and Therapeutic Tips

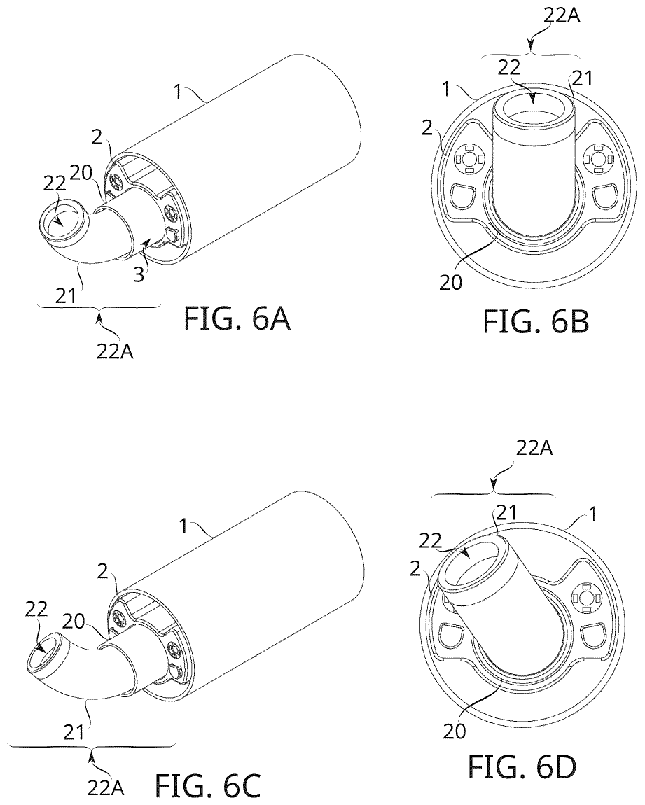

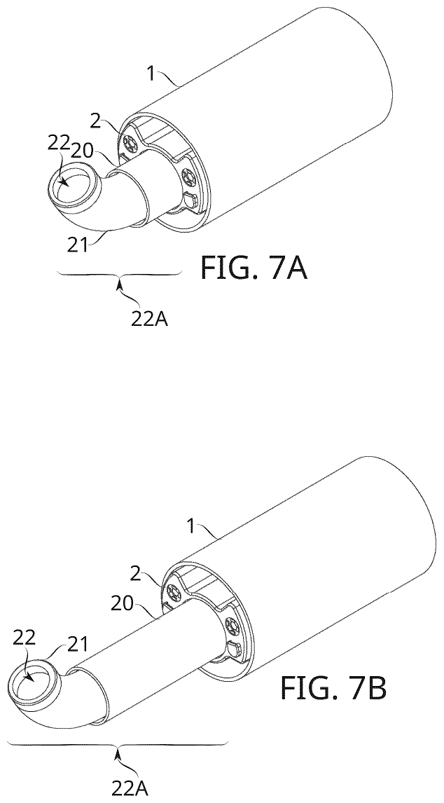

Reference is now made to FIGS. 5A-5D, which schematically illustrate a steerable working channel 22, according to some embodiments of the present disclosure. Further reference is made to FIGS. 6A-6D, which schematically illustrate rotation of steerable working channel 22 around the main working channel axis, according to some embodiments of the present disclosure.

Steering degrees of freedom include an angle of deflection sideways (away from a longitudinal axis of the channel), and/or an angle of rotation around a longitudinal (proximal-distal) axis. A maximum angle of deflection may be at least for example, 180°, 135°, 90°, 45°, or another angle. Optionally, the angle of rotation may be any angle around the proximal-distal axis.

FIGS. 5A-5B show endoscope 2 housing a main working channel 3. Outer channel tube 20 is passed distally via the main working channel 3. In turn, middle channel tube 21 passes distally via the outer channel tube 20. Middle channel tube 21 is configured to bend sideways, e.g., to an extent controlled from a proximal side of endoscope 2. Middle channel tube 21 may be elastically predisposed to bend upon leaving confinement of outer channel tube 20, and/or it may bend in response to direct bending control exerted, e.g., by adjusting tension on one or more elongated control elements extending proximally from middle channel tube 21 to a proximal end of endoscope 2.

Together, middle channel tube 20 and outer channel tubes 21 form components of steerable channel 22A. Middle channel tube 21 itself defines an inner working channel 22, thereby providing an adjustably positionable working channel terminus (distal aperture). Working channel 22 can be used to pass therapeutic and/or diagnostic tools distally, including positioning of these tools through a range of off-axis position. For example, FIGS. 5C-5C illustrate middle channel tube 21 with a bigger bend, according to control exerted by an operator.

FIGS. 6A-6B again show endoscope 2 together with an outer channel tube 20 passed via the main working channel 3. Middle channel tube 21 is passed further distally via outer channel tube 20 and bent sideways, for example as described in relation to FIGS. 5A-5D.

The bend is pointing in this configuration at 12 o'clock (upward) direction. FIGS. 6C-6D show a configuration with the outer channel tube 20 rotated counter-clockwise around its main axis, rotating middle channel tube 21 likewise. In this situation, middle channel tube 21 points to about the 10 o'clock direction, according to the rotation amount set by the operator.

It should be noted that middle channel tube 21 is optionally rotatable separately from endoscope 2 and/or outer channel tube 20, allowing, e.g., rotation of middle channel tube 21 while endoscope 2 and/or outer channel tube 20 remains stationary, rotation of endoscope 2 and/or outer channel tube 20 while middle channel tube 21 remains stationary, or rotation partially comprised of a plurality of these rotational motions.

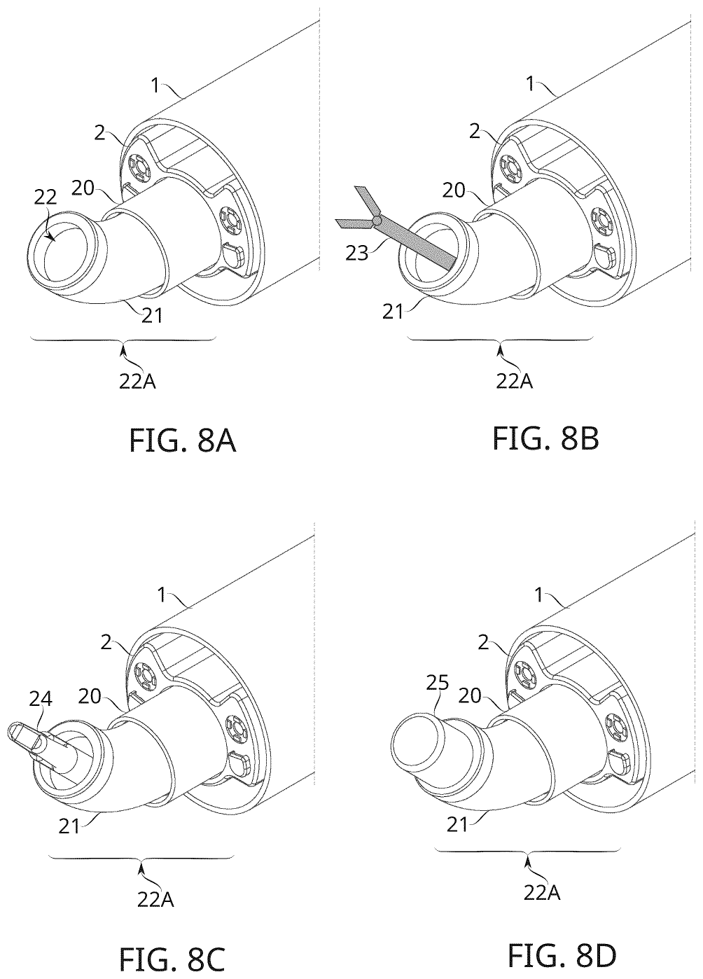

Reference is now made to FIGS. 7A-7B, which schematically illustrate an outer channel tube 20 operable to slide in and out from the main working channel 3 (i.e., operable to slide along a proximal-distal axis of main working channel 3). Middle channel tube 21 passes distally via outer channel tube 20, and is configured to bend sideways, for example as described in relation to FIGS. 5A-5D. Outer channel tube 20 moves linearly forward and backwards relatively to the main working channel 3 and endoscope 2, under control of the operator.

In any of these degrees of freedom, the operator optionally provides direct manual inputs (forces) to manipulate positions of steerable channel 22A (e.g., by operations performed on middle and/or outer channel tubes 21, 20). Optionally, movements of middle and/or outer channel tubes 21, 20 are performed at least partially under robotic control, e.g., robotic control to perform actions selected by the operator. The robotic control may be at any suitable level of automation. In some embodiments, all distances and angles of robotic movement are directly commanded by a human operator. In some embodiments, the robot is at least partially autonomous. For example, it may govern details of a single movement from a current position to a target position, or of a compound movement which visits several positions autonomously (e.g., to treat and/or measure a target).

Robotic control may act to modify commanded movements, e.g., movements performed and/or specified by an operator are limited, smoothed, and/or corrected according to suitable available parameters. The parameters optionally include, for example: limits on speed, acceleration, and or range of motion; machine-sensed (e.g., visualized) positions of targets and/or obstacles; and/or sensing of forces such as resistance to motion and/or pressure. The commanded movements modified may be exerted directly by manual user manipulation of the middle and/or outer channel tubes 21, 20, with robotic adjustments being superimposed on this, exerting guidance and/or countering forces/movements as necessary. Additionally or alternatively, robotic motions are performed substantially automatically, with manual adjustments optionally provided by the operator, e.g., to slow, speed, offset, countermand, and/or re-select automatic motions.

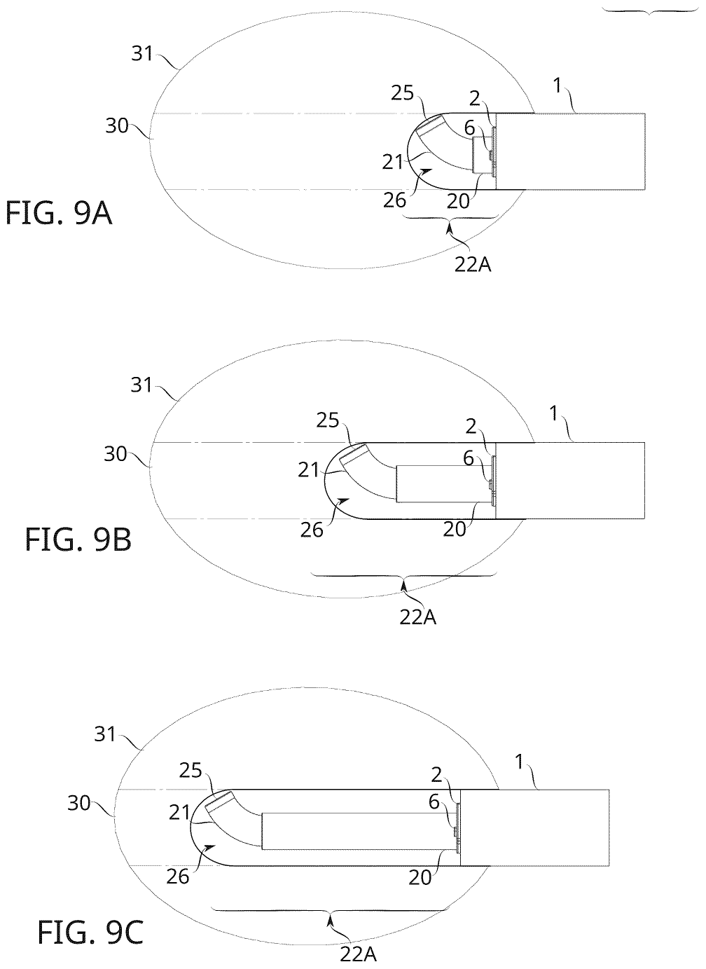

Reference is now made to FIGS. 8A-8D, which schematically illustrate tools with different tips that could be passed via the inner working channel 22 of steerable channel 22A, according to some embodiments of the present disclosure. The tool functions are optionally therapeutic, diagnostic, supportive (e.g, of illumination and/or positioning), and/or with another purpose.

FIG. 8A illustrates an open inner working channel 22, without any tools within. Such a configuration enables, for example, irrigation with water and/or or drugs into a cavity. Optionally this configuration is used to provide evacuation by natural flow, and/or by suction of fluids from the cavity to outside the body. This is referred to herein as “suction”.

FIG. 8B illustrates an inner working channel 22, with an off-the-shelf fixable endoscopic tool 23 passed distally through. The tool 23 may be used, e.g., for gripping, cutting, tearing, cauterization (e.g., electrical cauterization), or another purpose.

FIG. 8C illustrates an inner working channel 22 with an affixable bi-polar tool 24 passed distally through. Bipolar forceps may be manipulated, for example, to open/close, move in/out from the opening of inner working channel 22, and/or or rotate around its tip's main axis. Optionally, tool 24 is supported proximally by an adaptor sized to the lumen of middle channel tube 21 and which remains at least partially within middle channel tube 21 while tool 24 extends distally outside of it.

FIG. 8D illustrates an inner working channel 22 with a cutting tool 25 passed distally through. Cutting tool 25 optionally comprises, for example, a water jet cutting tool, an electrical current-based cutting tool, a laser-based cutting tool, an ultrasound based cutting tool (for example the CUSA) or a tool cutting by another means. In particular, cutting tool 25 is sized to fill the lumen of middle channel tube 21. Support this provides may confer potential advantages for positioning control and/or exertion of force.

Tools described in relation to FIGS. 8B-8D optionally themselves are actuatable to move in one or more degrees of freedom. For example, they move in to/out from the opening of inner working channel 22. Tools optionally rotate around the tool's longitudinal axis. Tools may be provided with additional degrees of freedom and/or actuation, according to their function. Optionally, tools are withdrawn and replaced without the need to remove the steerable working channel 22A itself. Movements may be controlled by the operator; optionally as manual manipulations, as robotic movements, and/or in conjunction with robotically performed movements, in any suitable combination, for example a combination as described for steerable channel 22A itself in relation to FIGS. 7A-7B.

In some embodiments, another tool is provided; for example, a tool carrying one or more cameras. In some embodiments, the camera tool itself is articulated, allowing adjusting of viewing angle, e.g., while working channel 22 remains stationary. In some embodiments, a therapeutic tool is provided with a camera additionally to its own working end. This is used, for example, to provide close inspection of regions selected by the positioning of steerable channel 22A. In some embodiments, a tool comprising electrodes for measurement and/or therapeutic delivery of energy is provided for use from within steerable channel 22A.

### Removing Large Tumors With a Retractor (L-Shape, I-Shape and Foldable Retractor)

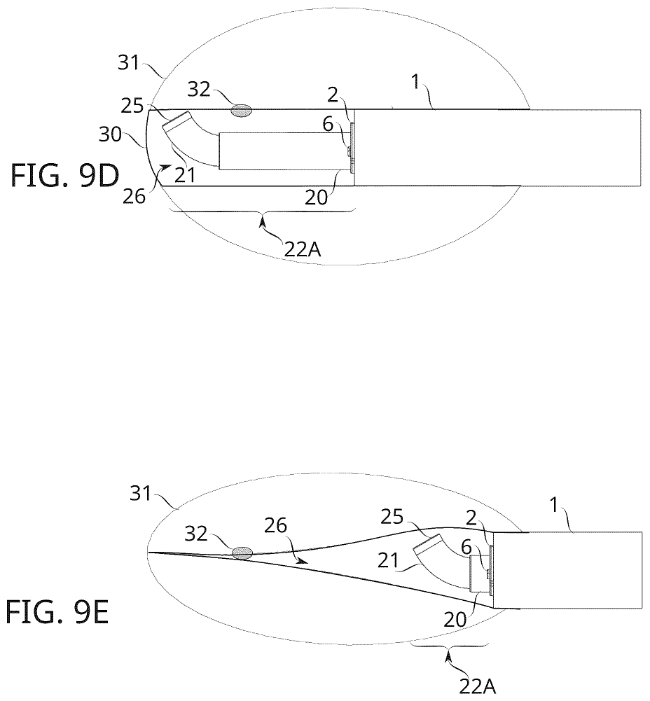

Reference is now made to FIGS. 9A-9E, which schematically illustrate operation of an endoscope 2 together with a steerable channel 22A within a region of body tissue, according to some embodiments of the present disclosure. In some embodiments, steerable channel 22A is used in positioning for treatment of a tissue portion and/or remove a tissue portion from the body. The tissue portion may comprise, for example, tumorous tissue. Examples herein of operations performed on a tumor or tumorous tissue (e.g., the examples of FIGS. 9A-11C) should be understood as not limited to such tissue; for example the operations may be performed on any kind of healthy tissue, unhealthy tissue and/or hemorrhage.

FIG. 9A illustrates introducer 1 housing endoscope 2, with a therapeutic tip 25 located at the tip of steerable channel 22A. Tip 25 is configured to remove tissue; in this example, tissue of a schematically represented tumor 31. Therapeutic tip 25 may comprise any tool or combination of tools and/or functions suitable for disrupting and removing tissue. Examples include mechanically-based (e.g., cutting and/or grinding), energy-based (e.g., using electricity, ultrasound, and/or laser light), and/or pressure-based (e.g., vacuum and/or jetting) tools for tissue disruption and/or removal.

Removal begins, in some embodiments, by operations within a working region 30 of tumor 31 located in front of (distal to) endoscope 2. As shown, working region 30 comprises an in-axis workspace of endoscope 2; that is, a volume extending distally from a distal end of endoscope 2, with about the same diameter as endoscope 2 and/or introducer 1. This is also referred to herein as a volume falling within the axial shadow of the endoscope 2 and/or introducer 1.

of elongated elements), and/or suitable to its use with respect to a target of treatment and/or diagnosis. For example, a practical usable length of the in-axis workspace beyond the introducer 1 is optionally equal or less than about 10 diameters of the introducer. It is noted though that introducer 1 may itself be advanced into the in-axis workspace in some embodiments, redefining the workspace, or in other terms, advancing the workspace along with it.

The full working space available from a given location of a distal end of endoscope 2 may be of larger diameter than the in-axis workspace, for example, as appropriate for the positioning, diameter, and/or radius of curvature of steerable channel 22A.

Nonetheless, there are potential advantages to using the in-axis volume extending distally from endoscope 2 as a preferred working volume. Accordingly, in some embodiments, initial operations to remove tumor 31 begin with clearance of a region of the tumor just large enough in diameter for introducer 1 and/or endoscope 2 to advance through until a distal side of the tumor 31 is excavated.

The option to advance endoscope 2 together with its optics provides potential advantages for visualization. Attempts to assess a target from a distance, e.g., from the viewpoint of lens 6, are potentially impeded when view of the target is excessively foreshortened, obscured by intervening material and/or lacking in detail for monitoring requirements. Conversely, confining operations to within the in-axis workspace may help keep therapeutic tip 25 and/or the surface of the region 26 which it is excavating in view. Close-up visualization may be impeded if the working region and/or wall of excavated space 26 extends too far radially outside the field of view provided through lens 6.

An in-axis working volume also provides potential advantages for support and/or control of positioning. In one potential support advantage, untreated tissue of tumor 31 is held substantially in its original position as introducer 1 and/or endoscope 2 advance, preventing its uncontrolled collapse or other movement. In another potential support advantage, therapeutic tip 25 is optionally kept within a short distance of the axial support provided for it by outer channel tube 20, endoscope 2, and/or introducer 1. This potentially provides greater positioning reproducibility of tip 25 in response to operator inputs (e.g., prevents flexing). It also potentially helps to maintain certainty as to what tissue is being accessed. For example, with greater radial working distance, there may be an increase in uncertainty about what original tissue is actually being accessed, as tissue compliance leads to compression and/or collapse to uncertain degrees. The support provided by introducer 1 and/or endoscope 2 may not be as tightly controlled if the initially excavated area is larger than their diameter.

With reference also to the potential advantages of maintaining an in-axis working volume: in some embodiments, operators are provides with warnings and/or indications when therapeutic tip 25 exceeds and/or is at risk of exceeding the boundaries of working region 30, at least during an initial phase of the procedure. The warnings and/or indications may be based, for example on sensing (e.g., encoder-based sensing) of the current control state of steerable channel 22A. In some embodiments, additionally or alternatively, direct sensing of the position of steerable channel 22A is performed. For example, display of an imager's field of view may be overlaid with indications of positions within or outside of the in-axis working volume. This is optionally indexed to data describing the relative positions of the imager and therapeutic tip 25 along a proximal-distal axis. This may be derived from control state, and/or from the apparent size of steerable channel 22A and/or some part of it in the imager's view.

Between the examples of device positioning shown in FIGS. 9A-9E, therapeutic tip 25 is moved according to operator and/or robotic control to reach remove tissue for removal, according to movements of outer tube 20, middle tube 21, endoscope 2, and/or introducer 1. For example, outer channel tube 20 advances distally (forward), while middle tube 21 bends to a selected amount away from a proximal-distal axis of endoscope 2 and/or rotates around this proximal-distal axis. Examples of configurations in various stages of excavation and/or longitudinal advance are shown in FIGS. 9A-9D. The extent of excavation is indicated as excavated region 26. Variations in curvature and/or rotation of middle tube 21 are performed during advance to move clear out tissue in circumferential and/or radial positions other than those adjacent to the upward-facing positions of therapeutic tip 25.

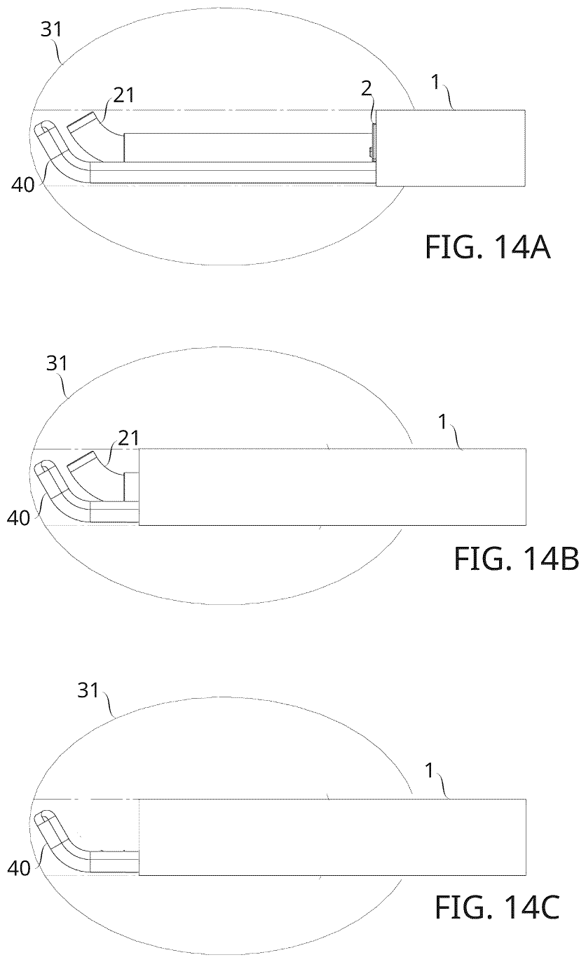

States corresponding to complete removal of the tissue originally located in the endoscope's in-axis workspace 30 are shown in FIGS. 9D-9E. The tissue in the endoscope's off-axis workspace optionally remains untreated at this point, reserved for later treatment (e.g., as described in relation to FIGS. 14A-14F).

In FIG. 9D, introducer 1 and endoscope 2 are shown further advanced into tumor 31, with the effect of bringing lens 6 (and its associated imaging device) distally for closer monitoring of the operations of therapeutic tip 25, e.g., target 32. This also has the effect of providing support to tissue in the excavated region.

FIG. 9E illustrates a condition resulting from a backward (proximal) movement wherein the user decides to move the therapeutic tip 25, endoscope 2, and introducer 1 back to the location of, e.g., FIG. 9A. Once tip 25, middle tube 21, outer tube 20, endoscope 2 and introducer 1 are not occupying the endoscope's in-axis workspace 30, the tumor may collapse inward. Collapse may happen for any one or more of several reasons. Examples include: pressure applied by the remaining off axis tumor 31, gravity forces applied on the tissue, intra-cranial pressure, tissue swelling, and/or water accumulated in surrounding tissue. Optionally, pressure which postpones collapse is provided during excavation, e.g., by insufflation and/or irrigation, but at a certain point it may be determined that this cannot be safely and/or controllably continued. Regardless of safety/control issues, collapse may optionally be encouraged by discontinuing fluid administration, changing a balance of fluid administration and fluid removal, and/or by applying suction.

The collapse may be useful in some aspects, e.g., because it brings new tissue into the working region 30. Potentially, tissue is enters region 30 in a measured amount, since, e.g., it is self-limited by the diameter of the excavated region 26. Collapse may bring some surface regions of the excavated volume 26 into clearer (e.g., more frontally direct and/or less foreshortened) view of lens 6.

However, viewing at least more distally is potentially impeded by collapse. For example, a target 32 that easily viewed from the position of lens 6 in FIG. 9E is obscured in FIG. 9E due to the inward collapse of excavated region 26 into the endoscope's in-axis workspace 30. In the region just distal to introducer 1 and/or endoscope 2, the distance of collapse may be smaller. This may allow close-in operations to continue tissue removal with therapeutic tip 25. If an operator finds that they prefer to operate tip 25 at a particular distance from lens 6, endoscope 2 may be withdrawn into introducer 1 while introducer 1 acts to prevent collapse.

However, working conditions as illustrated for the situation illustrated in FIG. 9E may still be subject to unwanted visual interference and/or lack of control. Collapsed tissue intrudes from all radial sides. Furthermore, there is no support provided distal from therapeutic tip 25. This may make it difficult to perform certain operations. For example, withdrawing tip 25 to inspect the surface of a region it is both currently treating and supporting may result in movements of the surface which make it more difficult to see and/or identify. Returning of tip 25 to renew work may be difficult to perform reproducibly, and/or with full confidence that tissue removal work will begin in the same area as it was previously operating on.

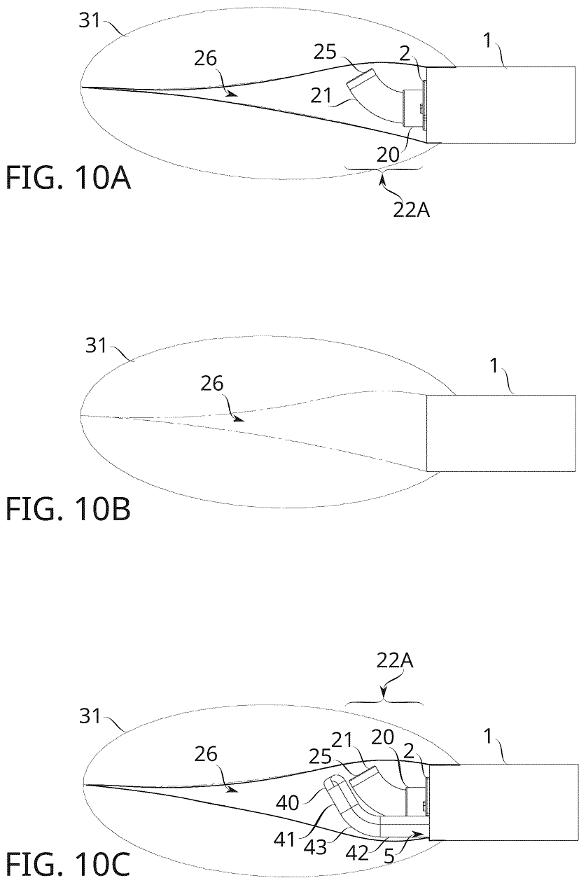

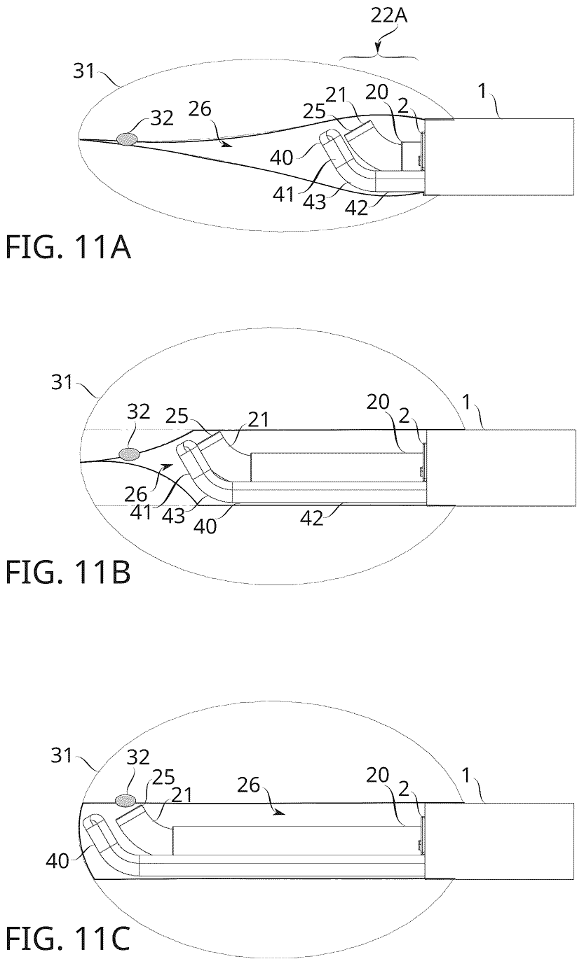

Reference is now made to FIGS. 10A-10C and 11A-11C, which schematically illustrate the use of a retractor 40 together with a steerable channel 22A, according to some embodiments of the present disclosure.

FIG. 10A illustrates endoscope 2 within introducer 1, and withdrawn to a proximal side of tumor 31. This corresponds, for example, to the situation also shown in FIG. 9E. Optionally at this stage, endoscope 2 is removed for example, as shown in FIG. 10B. This clears introducer 1 to allow introduction of retractor 40 along its lumen. With endoscope 2 removed, distal wall 41 of retractor 40 can be introduced even though it may extend across most or all of the lumenal cross-section of introducer 1, or at least extend so that it intrudes into regions otherwise occupied by the cross-section of endoscope 2. Endoscope 2 is then re-introduced.

A base 42 of retractor 40 extends proximally from distal wall 41. In cross-section, it is sized and shaped to occupy one of the compound working channels defined between endoscope 2 and introducer 1, for example, compound working channel 5, e.g., as described in relation to FIGS. 2A-2B. Optionally, it fully occupies at least a circumferential extent of compound working channel 5 for at least a portion of its longitudinal extent extending proximally from distal wall 41. This optionally comprises a large portion of a circular circumference, for example, about ⅓ or more of a circular circumference. In some embodiments, it extends at least about 20% around a circumference centered on the central axis of the introducer 1, or at least about 40% around this circumference.

In some embodiments, a region 43 connecting distal wall 41 to base 42 is flexible, and distal wall 41 itself is sized and shaped so that when region 43 is straightened (e.g., when constrained by confinement within compound working channel 5), distal wall 41 can pass along compound working channel 5 even with endoscope 2 remaining in place. For example, the distal region of retractor 40 providing the bent region of the “L” (that is, distal wall 41 and region 43) may initially be stowed folded flat while within introducer 1, and then bend (e.g., elastically) into position upon advancement out of introducer 1. Upon retraction, it folds straight again under constraint by the introducer 1 and/or endoscope 2.

In effect, this amounts to the configuration of region 43 to act as a hinge between base 42 and distal wall 41. In some hinging embodiments, restoration of distal wall 41 to its angled position may occur upon operation of a control element (e.g., elongated tensioning element such as a wire) that locks region 43 into shape, or via another mechanism.

A fixed shape of the distal region of retractor 40 comprising region 43 and distal wall 41 nevertheless provides potential advantages, e.g., for simplicity of construction, case of passage, and/or selection of construction materials. For example, retractor 40 may be constructed entirely of a single piece of stainless steel. At least along the longitudinal extent of base 42, this may comprise of a sheet of stainless steel which is relatively thin compared to the height of compound working channel 5, e.g., about 1 thick out of 4-5 mm total height provided. This potentially leaves the lumen of compound working channel 5 open to perform another function, such as irrigation, suction, tool passage, or another function.

In some embodiments, the sheet is also curved, for example, curved to follow a radially outer contour of channel 5. This provides potential advantages for stiffening of retractor 40.

Base 42 is not limited to be flattened, to be circumferentially solid, or to fill channel 5 circumferentially. For example, base 42 may comprise one or more support rods from which distal wall 41 extends. Additionally or alternatively, base 42 may defines apertures along its longitudinal and/or circumferential extents, e.g., apertures of a mesh, and/or holes in a solid sheet of material. Along some or all of its extent, base 42 may itself define one or more internal channels (i.e., it may be hollow), and/or one or more channels which it defines along with a portion of the outer wall of endoscope 2, and/or a portion of the inner wall of introducer 1. Base 42 may comprise a tubular body with apertures along it, for example, to allow longitudinally and/or circumferentially distributed administration of irrigation fluid and/or suction.

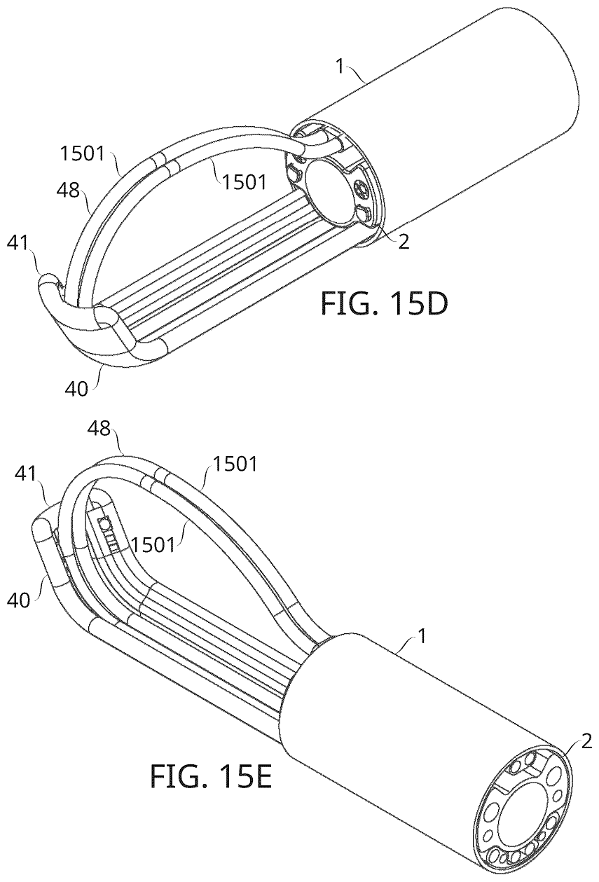

Similarly, distal wall 41 and/or region 43 may comprise portions of a curved flat sheet, or another shape, for example as described for base 42. An example of a distal wall 41 configured with an aperture 41d is shown, for example, in FIGS. 17A-17B. In some embodiments, edges of distal wall 41 are rounded over (e.g., given a smooth profile), in order to avoid damaging tissue as distal wall 41 advances distally, e.g, through at least partially collapsed tissue. Distal wall 41 may be solid over at least 60% of its surface.

FIGS. 10A-10C and 11A-11C illustrate an L-shape retractor design, but the several other retractor designs could be used with such system. For example, it should be understood that portions of retractor 40 which are not to be withdrawn proximally past the distal end of endoscope 2 after endoscope 2 is re-introduced are optionally of any cross-sectional shape which fits within introducer 1. Portions which fit within introducer 1 but not within the lumen of compound working channel 5 formed upon the re-introduction of endoscope 2 are referred to herein as “oversized portions”. They are oversized in the sense that they are too large to be withdrawn via the compound working channel along which they connect to regions on a proximal side of introducer 1. Oversized portions be oversized to any extent, e.g., up to the extent that they comprise an entire circumference of a cylindrical wall having an outer radius with the inner radius of introducer 1. The cylindrical wall is optionally provided with one or more cutouts (e.g., slots and/or apertures) through which a tool such as therapeutic tip 25 can reach tissue. In another example, distal wall 41, optionally comprises a disk, annulus, or other shape which entirely spans the diameter of inner lumen of introducer 1.

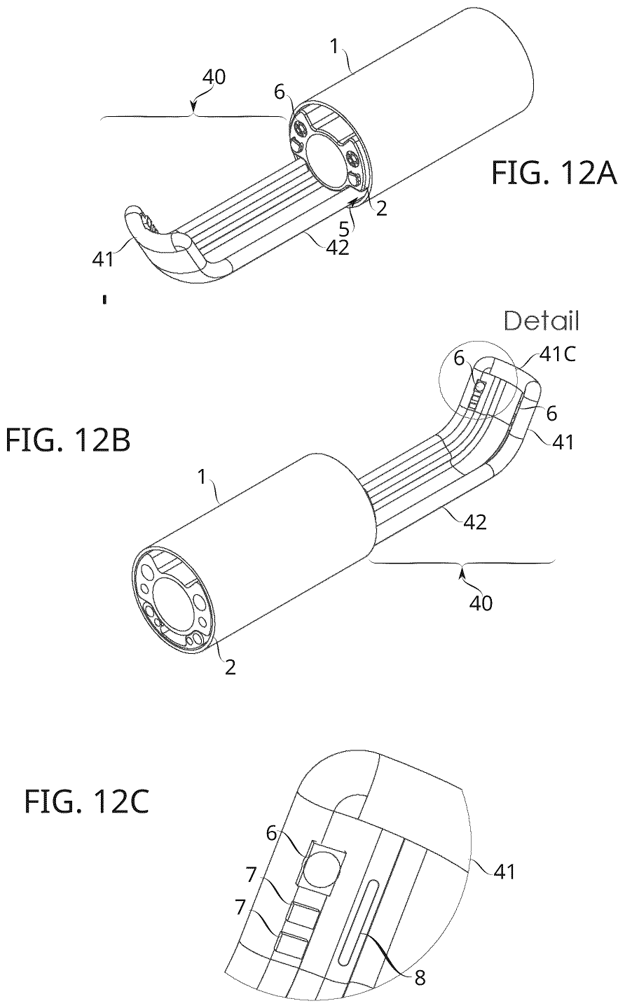

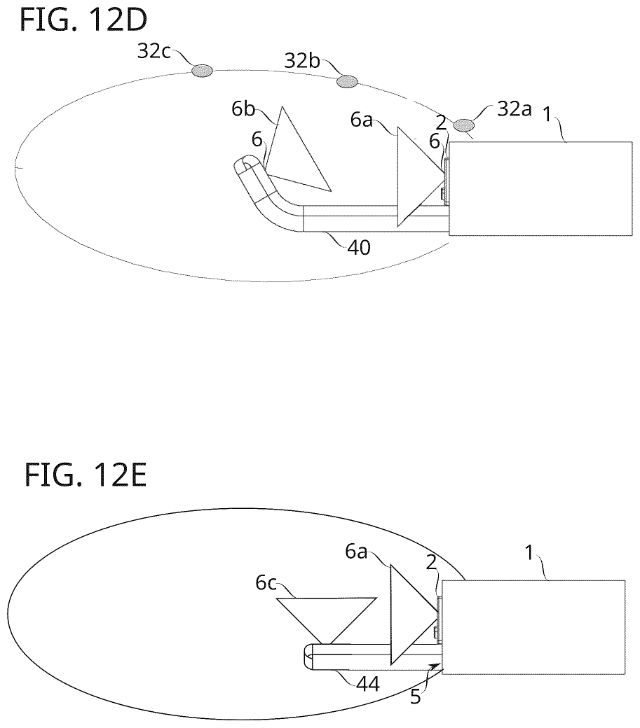

As shown, e.g., in FIG. 10C, oversized portions optionally block distal advance of steerable channel 22A beyond them. However, oversized portions may comprise a central hollow and/or aperture sized to allow steerable channel 22A to pass distally past some or all of the oversized portion. Furthermore, it should be noted that the length of oversized portions need not be as longitudinally short as shown for distal wall 41 plus region 43 in FIGS. 10C-12C. For example, withdrawing endoscope 2 by some distance allows longer extents of an oversized portion to be withdrawn by a similar additional distance into introducer 1. Conversely, introducer 1 may be advanced relative to the other system elements, e.g., in order to provide tissue support (e.g., as described in relation to FIGS. 14A-14C). However, increasing the longitudinal offset between the working portion (e.g., tip 25) and imagers (e.g. via lens 6) of endoscope 2 has the potential to interfere with monitoring of the placement and/or operation of tip 25. In some embodiments, monitoring elements such as one or more cameras are provided as part of retractor 40, e.g, as described in relation to FIG. 12D.

It is noted, furthermore, that distal portions of retractor 40, whether oversized or not, optionally expand, deflect are expandable and/or are deflectable away from a longitudinal (proximal-to-distal) axis of introducer 1 after they leave introducer 1, for example as described for embodiments wherein region 43 acts as a hinge. The connection between base 42 and distal wall 41 is not necessarily at only one edge of distal wall 41 (that is, to form an “L” shape.). For example, distal wall 41 may be attached to retractor 40 via top and bottom bases 42 (e.g., bases extending from different compound working channels), forming an I-shape. Attachment may be flexible so that relative adjustment of the distal advance of the bases changes angle between either horizontal arm (the bases 42) and the angled arm (the distal wall 41).

Distal wall 41 may be shaped to introduce radial deflection in the position of a tool advanced sufficiently along compound working channel 5 to reach it. Distal wall 41 may include a portion that deflects such a tool to the extent that the tool extends proximally backwards from distal wall 41.

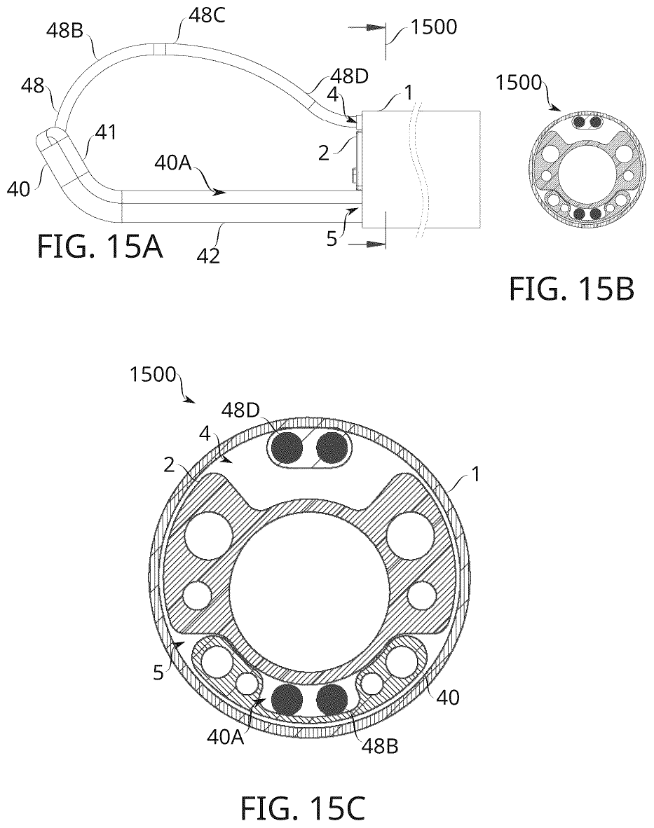

Any of base 42, distal wall 41, and region 43 optionally support one or more attached elements. An attached element may comprise, for example, a camera (e.g., as described in relation to FIGS. 12A-12E), another imager type such as an ultrasound imager (e.g. as described inn relation to FIG. 16B), and/or an illuminator. A camera, imager and/or illuminator may configured to provide one or more supplemental fields of view, e.g., into the working volume, e.g., of steerable channel 22A.

The camera and/or illuminator may be configured to support highly magnified/short working distance (e.g., microscopic) examination. For example, (e.g., if provide facing radially outward), they may allow examination of the fine structure of the excavated wall, for example to help determine if there is remaining tumor or other targeted tissue to address. Optionally (e.g., facing radially inward), the tissue samples can be brought to it by another tool.

Additionally or alternatively, an attached element may comprise another tool for use in operations of the procedure. For example, the attached element may comprise a nozzle used to clean other elements (e.g., by washing them with fluid). The attached element may comprise an electrode useable to test impedance characteristics of the tissue environment and/or of samples brought to it.

The attached element may comprise one or more needles and/or nozzles that administer a substance used in the procedure, by flooding the working space, and/or by injection onto and/or into selected regions of the surrounding tissue. In some embodiments, the substance is therapeutic in action, e.g., it induces or prevents blood clotting, induces cellular death (e.g., to “finish” a tissue surface after mechanical treatment in case of a risk of residual contamination with tumorous material), or has another therapeutic effect. Optionally, the administered substance helps to track the progress of mechanical removal of tissue. For example, it comprises a selective (e.g., antibody-based) or non-selective stain. A selective stain may help reveal whether targeted tissue remains and/or whether non-targeted tissue is exposed. Whether selective or non-selective, tissue stain May be administered to a selected stain size and/or depth of staining, and/or in patterns which can be sensed (e.g., visually inspected) as more tissue is removed to help determine where and/or how much tissue has been removed. Optionally, staining is used to help mark (e.g, enhance the visual contrast of) regions that should be avoided by treatment operations. Optionally, staining is used to establish landmarks which assist in modelling changes in tissue shape as a procedure progresses.

FIGS. 11A-11C illustrate forward (distal-ward) movement of retractor 40. Forward movement of retractor 40 re-opens the in-axis workspace. At least on a dorsal (e.g., circumferentially outward-facing) aspect of base 42, a wall of excavated region 26 is supported at about its original radial position. A distal tip of distal wall 41 provides distal support of tissue to the extent of its reach.

As shown, distal wall 41 reaches a point most of the way across the inner diameter of introducer 1, from the position of the dorsal aspect of base 42. The example shown is about 87% of the inner diameter of introducer 1, and about 83% of the outer diameter.

Optionally, distal wall 41 is longer, e.g., extending all the way across the inner diameter of introducer 1. For example, if moveable relative to base 42 (e.g. if region 43 is flexible) distal wall 41 may extend even further, for example all the way to and optionally beyond the outer wall of introducer 1. In some embodiments, a distal part of base 42 includes a radially outward deviation (i.e., a deviation toward the bottom of FIGS. 11A-11C). The deviation is shaped so that when a more proximal part of base 42 is positioned within the constraints of compound working channel 5 (e.g., after re-insertion of endoscope 2), the deviated part is positioned radially outward of the inner radius of introducer 1. For example, it is positioned at the outer radius of introducer 1, and optionally further. With these or similar modifications to the example shown, introducer 40 may be configured to fully extend across the outer diameter of introducer 1, at least for nearby locations along its longitudinal extent.

In some embodiments, one or more additional retractors are used to provide additional support, e.g., passed through introducer 1 while endoscope 2 is withdrawn, and sized and shaped to occupy a portion of compound working channel 4, compound working channel 5, or another working channel outside of endoscope 2 itself once endoscope 2 is re-introduced. Distal to endoscope 2, the one or more additional retractors may be shaped in any suitable fashion, for example as described in relation to retractor 40, to provide and/or augment features also described in relation to retractor 40.

Even without support that fully crosses the outer diameter of introducer 1, the previously collapsed tissue may retain sufficient elasticity such that when forced by retractor 40 into an at least partially open state, it tends to self-restore, at least in part, to a still more fully opened state. Additionally or alternatively, forces that induced the original collapse of the excavated region 26 might be transitory, so that upon re-opening by the retractor 40, an uncollapsed state is restored.

Upon re-opening of collapsed excavated region 26 (e.g., to its original size, or to another size), a region such as target 32 that is hardly seen in FIG. 11A is more easily seen, allowing it to be accessed to cut away or otherwise interact with, e.g., as shown in FIG. 11C.

Reference is now made to FIGS. 12A-12D, which show a perspective view of an L-shaped retractor 40, according to some embodiments of the present disclosure.

FIG. 12A illustrates an L-shape retractor 40 wherein the lower part of the L-shape (base 42) has a cross section that fits in the lower compound working channel 5, located between the introducer 1 and the endoscope 2. Being positioned this far radially from the center of introducer 1, the lower part of the L-shape (base 42) pushes tissue downwards as it extends, nearly to the outer circumference of introducer 1.

The forward angling of the distal part of the L-shape (distal wall 41b) helps retract collapsed tissue forward and downward.

FIG. 12B illustrates L-shape retractor 40 from the other side. Lenses 6, mounted near the tip 41c of distal wall 41 provide optics for cameras. Fields of view of the cameras are oriented to inclusion regions generally on their proximal side (e.g., viewing backwards).

FIG. 12C shows a zoomed detail of the lenses 6 and the light sources 7. Optionally, light sources 7 are operated on a duty cycle which alternates (e.g., stroboscopic ally) with exposure periods of camera elements pointed at them, e.g, pointed at them from the vantage of the distal end of endoscope 2. Optionally, light sources 7 are hooded and/or directional to avoid dazzling camera detectors opposite them (e.g., camera detectors housed in endoscope 2). Optionally, camera detectors opposite light sources 7 are masked in the region pointed at light sources. The optics are optionally washed via the washing nozzle 8 located nearby and pointed at the optics 7.

FIG. 12D shows a side view of an L-shape retractor 40. Fields of view 6a and 6b represent camera fields of view from positions of lens 6 (and associated camera detector) on endoscope 2 and retractor 40, respectively. Fields of view could be wider or narrower than shown. Along a proximal-to-distal axis, the cameras are pointed in opposite directions: one forward and one backwards. This configuration has potential advantages for providing redundancy and/or greater coverage in the case that tissue intrusion or another field of view limitation blocks or otherwise prevents a view from one of the camera directions. For example, region 32c is viewable from the endoscope's field of view 6a, but not target 32a. If target 32a for example, starts to bleed, an operator with a view only through field of view 6a will not be able to spot such bleeding. However, bleeding target 32a is within proximally-oriented field of view 6b.

Target 32b can be seen from either direction, potentially providing redundant viewing and/or enhancing visual information provided about the target.

Reference is now made to FIG. 12E, which show a shows a straight-base retractor 44, according to some embodiments of the present disclosure. Retractor 44 optionally has no oversized portions, allowing it to slide in and out of the lower compound working channel 5 without the need to remove endoscope 2 temporarily from introducer 1. Retractor 44 optionally is provided with upward looking cameras field of view 16c. Optionally, it is a plain retractor with the same shape but without any cameras.

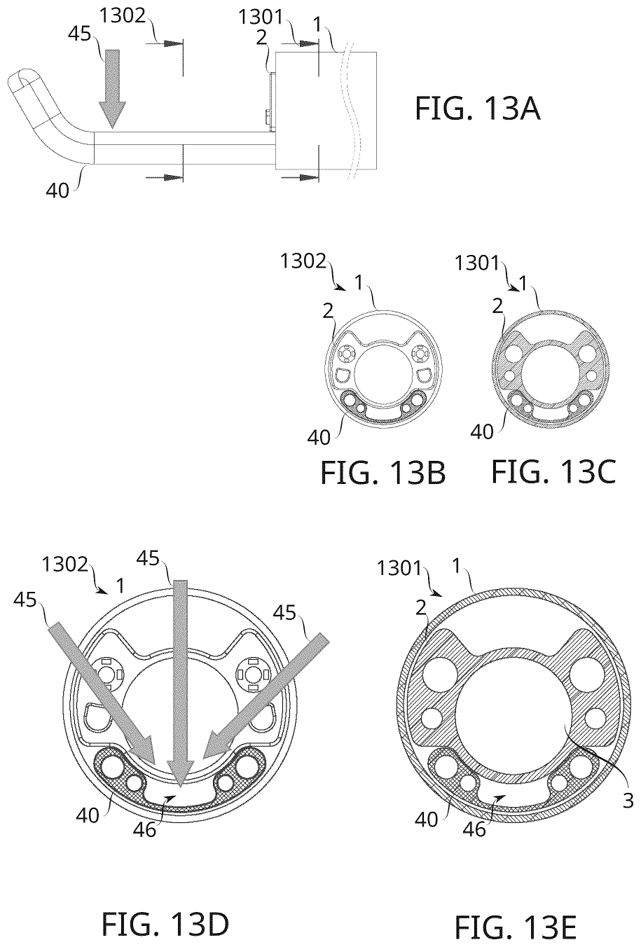

Reference is now made to FIGS. 13A-13E, which schematically illustrate an L-shape retractor 40, according to some embodiments of the present disclosure. FIG. 13A shows a side view. FIG. 13B shows cross section 1302 of FIG. 13A, and FIG. 13C shows cross section 1301 of FIG. 13A. FIG. 13D shows cross section 1302 in more detail, and FIG. 13E shows cross section 1301 in more detail.

In the side view of FIG. 13A, arrow 42 indicates fluids and waste flowing into the workspace. These are to be evacuated outside the body.

In the detailed cross-section of FIG. 13D, inflows 45 of fluids and waste reach the cavity 46 defined by retractor 40. Upon a suitable pressure difference and/or gravity gradient being established relative to a proximal side of compound working channel 46 (FIG. 13E), the inflows 45 flow proximally through it. Compound working channel 46 is formed between the endoscope 2 and the retractor 40. The retractor 40 and endoscope 2 are held in place against each other by enclosure within introducer 1.

### Removing Large Tumors With a Retractor and Deployable Scaffold

Reference is now made to FIGS. 14A-14F, which schematically illustrate deployment of a scaffold 48 allowing and assist in keeping a clear workspace in the tumor, according to some embodiments of the present disclosure. Potentially, the scaffold arrangement helps with maintaining the shape of tumor 31, at least in currently active regions of tissue removal.

in some embodiments, scaffold 48 comprises a pair of wire elements extending parallel to each other. Deployable scaffold 48 is made, for example, from elastic materials which accept a spring temper (for example nitinol, stainless steel or other materials). The material may be bent to a specific formation selected to keep the tissue supported in a suitable shape for working on. Optionally, more than one shape of scaffold 48 is provided, e.g., as different shapes along different parts of its length, and/or as wires differently bent. For example, a single scaffold 48 may be tempered to a range of different radii of curvature along its length, with the tendency to curve more or less tightly being used to adjust the shape of the upper loop portion 48A.

FIG. 14A shows retractor 40 and middle tube 21 upon reaching the distal end of tumor 31. In FIG. 14B, introducer 1 is advanced forward.

In FIG. 14C middle tube 21 and endoscope 2 are removed. Introducer 1 supports temporarily the remaining off-axis portion of tumor 31.

FIG. 14D shows a deployable scaffold 48, inserted from outside the body, and passing distally via upper compound working channel 4. Encountering distal wall 41 of retractor 40, it bends downward, then proximally back along retractor 40. It passes proximally via remaining space of lower compound working channel 5. Continued advancing of scaffold 48 through its loop results in it exiting the body, e.g., exiting a proximal side of introducer 1, at which point it may itself become useful for controlling (e.g., steering and/or advancing/retracting) scaffold 48. upper loop portion 48A expands laterally to support remaining tumor tissue. Optionally, scaffold 48 is constructed with a relatively flexible distal portion which is easily deflected by distal wall 41, followed by an increasingly stiff region (gradually or step-wise stiffer), which may be more suitable to provide tissue support. The distal side of scaffold 48 may comprise, e.g., an atraumatic tip. Optionally, tools advanced through introducer 1 may be used grab on to scaffold 48 and assist its correct deployment. Optionally, scaffold 48 is fitted to retractor 40 when retractor 40 is first advanced distally, and fed through introducer 1 along with retractor 40.

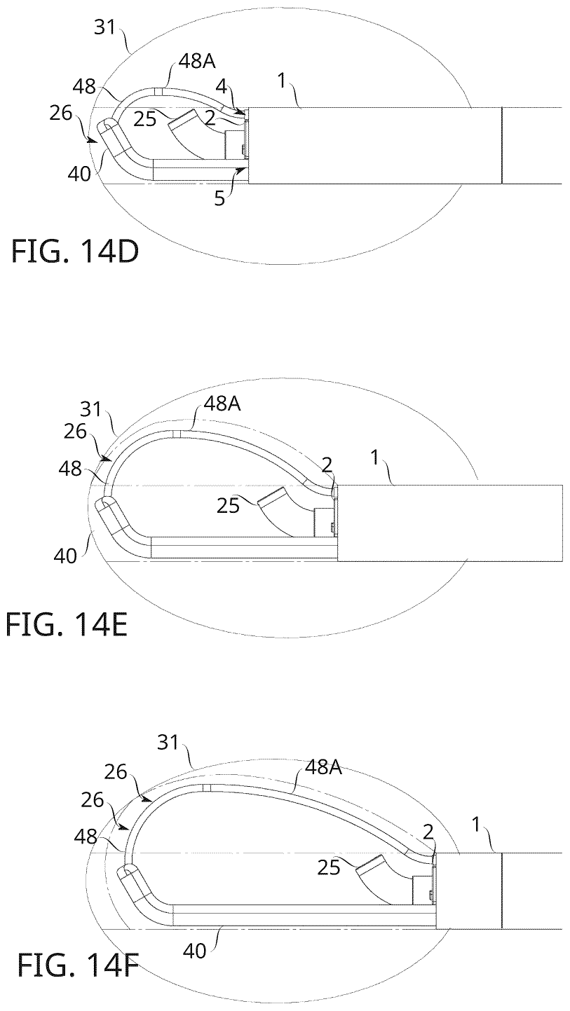

FIGS. 14E-14F illustrate that while moving endoscope 2 and the introducer 1 backwards (proximally) the user can keep the upper loop portion 48A of the deployable scaffold in place, and even steer it. Steering comprises, for example, manipulating tension on the loop, rotating endoscope 2, and/or controlling the relative position of retractor 40 (distally) and introducer 1 and/or endoscope 2 (proximally) along the proximal-distal axis. Steering of embodiments of loop 48 comprising a plurality of wires is described, for example, in relation to FIGS. 15D-15E, herein. At successively expanded positions of upper loop portion 48A, therapeutic tip 25 is operated to expand the excavated region 26, e.g., by removing tissue alongside loop portion 48A and/or tissue which intrudes past it.

Optionally: with suitable balancing expansion of loop portion 48A with removal of tissue, tumor 31 potentially retains something close to its original shape, at least in the direction that is currently supported by loop portion 48A. This provides a potential advantage insofar as tumor 31 may have been previously imaged, and a surgical plan devised for manual and/or robotic implementation that relies on the tumor size and position that was determined to exist.

In some embodiments, locations along scaffold 48 are provided with fiducial markings e.g., scoring, coloration, radio-opaque inclusions, surface modifications affecting ultrasound-reflectance (e.g., flattening and/or roughening), or another type of mark. Imager outputs may be used to detect the fiducial markings, allowing estimation from their known configuration and their image appearances of the current shape and/or position of the scaffold 48. This can help, for example, to assess the volumetric extent of a current excavated region. Optionally, scaffold 48 is rotated to a plurality of locations around the proximal-distal axis, shape assessed at each location helps to determine the overall size and/or shape of the excavated volume.

In some embodiments of the present disclosure, scaffold 48 is configured to assess the excavated region based on how it makes contact with tissue surfaces, e.g., pressure and/or electrical contact. This information may help, for example, to assess excavating progress, and/or identify/characterize a degree of tissue swelling.

In some embodiments, deformations of scaffold 48 from one or more reference shapes are used to assess internal pressures, e.g., to distinguish tissue that scaffold 48 is compressing from tissue that it is simply lying against. Optionally, scaffold 48 is advanced or withdrawn (e.g., fed through its loop via introducer 1 by advancing one side and retracting the other) to place different portions of itself within upper loop 48A. The different portions optionally comprise a range of differently constructed regions, e.g., with different fiducial markings, different resistances to deformation, and/or differences in another property. The differences are optionally used to help assess, e.g., the pressure and/or geometry of the currently exposed tissue surface. For example, determining the weakest part of scaffold 48 that does not noticeably deform may provide an indication of pressure. A portion of scaffold 48 may be provided with a contact electrode (e.g.), a metal region free from an insulating coating). This may be used to assess contact quality (e.g., as an indication of contact pressure) and/or electrical properties of contacted tissue, such as its dielectric properties.

In some embodiments, scaffold 48 is used together with a pattern of structured light to help assess its shape and/or interaction with tissue. For example, scanning bands of laser light are projected along its longitudinal extent, and their positions imaged. Light band positions and/or spacing as projected on to the imager's imaging plane shift depending on the location in depth of the surface probed.

Scaffold 48 may be used to mark tissue, e.g., to help track excavation progress. In some embodiments, a region along the longitudinal extent of scaffold 48 is loaded with a staining material (e.g., a dye contained within one or more scored recesses). To administer the staining material, the region can be dragged through the region of contact with tissue (e.g., upper loop 48A). Staining has potential advantages to assist machine vision extraction of features in the procedure environment, e.g., by increasing contrast and/or introducing features which are readily identifiable spectrally (e.g., a color and/or fluorescence/phosphorescence). Optionally, any of illuminators 7 is configured to produce one or more wavelengths that induce fluorescence and/or phosphorescence in the stain used, and/or which emphasize contrast of the stain with it surroundings.

In some embodiments, RF energy delivered to an electrode portion may be used to create a small ablation which is available thereafter as a reference (at least until it is excavated). Additionally or alternatively, RF energy lesioning is used to cauterize bleeding regions.

Marks may have a characteristic depth (e.g., a depth of lesioning and/or a depth of staining) which helps to assess excavation progress as the lesion is removed. Optionally, marks are used to help return scaffold 48 and/or other elements such as excavating tools to previous positions in a more deterministic fashion, e.g., the marked position itself, or another position having some particular relationship to the marked position. Movements of marked areas are optionally used to help assess tissue shifts and/or deformations, e.g., by comparing the shape and/or position of the tissue when is was marked to the current shape and/or position of the tissue. Optionally, a plan of how tissue is to be removed is adjusted based on the observed movements, e.g., adjusted automatically by deformation of a model of the region targeted for treatment to match movement and/or shape observations.

Optionally, marking is performed using a selective stain; e.g., a tagged antibody stain that preferentially labels healthy or unhealthy tissue. This may assist in assessing whether excavation progress has reached a region of healthy or otherwise non-targeted tissue.

Reference is now made to FIGS. 15A-15E, which schematically illustrate an introducer 1 comprising an L-shaped retractor 40 together with a deployable scaffold 48, according to some embodiments of the present disclosure. Herein, the term “L-shaped” in reference to a retractor 40 refers to a shape which includes a relatively long and straight portion leading to a curved portion; and another portion, optionally straight, which follows the curved portion, and is shorter than the relatively long and straight portion. The curved portion does not necessarily curve a full 90°; for example, it may curve at least 30°, at least 45°, or at least 60°. More particularly, FIG. 15A presents a side view. FIGS. 15B-15C show cross sectional views from the region marked by cross-section plane 1500 in FIG. 15A. FIGS. 15D-15E show perspective views.

In some embodiments, scaffold 48 comprises a first loop part 48D, and a second loop part 48B, which join each other through a third loop part 48C. Loop parts 48D and 45B each extend from introducer 1 from different sides of endoscope 2. In some embodiments, first loop part 48D extends out of compound working channel 4 which is optionally sized large enough that it leaves room also for a working tool (not shown, but optionally configured for performing operations such as described also for working tools used with working channel 22) to occupy the working channel at the same time as first loop part 40D.

In some embodiments, L-shaped retractor 40 extends out of introducer 1 along compound working channel 5, left open as a space between the cross-sectional shape of endoscope 2, and the interior lumenal wall of introducer 1.

Furthermore, in some embodiments, L-shaped retractor 40 comprises a guiding channel 40A, along which first loop part 48B extends. L-shaped retractor 40 is stiff enough to maintain the shape of its straight extended portion (base 42) and distal angled portion (distal wall 41), despite pressure from scaffold 48 as scaffold 48 is extended further and further from introducer 1. This causes scaffold 48 to eventually bulge through first loop part 45D and/or a portion of third loop part 45C.

In some embodiments (FIGS. 15D-15E), scaffold 48 comprises one or more wire struts 1501, for example, two wire struts 1501 as shown in FIGS. 15A-15E. In some embodiments, 3, 4, or more wire struts 1501 are provided. Even with the wire struts 1501 considered together, scaffold 48 is optionally rather narrow, e.g., about 1 mm across or less.

In some embodiments, scaffold 48 is steerable. Examples of steering manipulations include: advancing one of the two struts 1501 to a different distance than the other (introducing a torsion); rotating endoscope 2, L-shaped retractor 40 and scaffold 85 relative to introducer 1; changing the overall size of the loop of scaffold 48 by advancing and/or retracting wire struts 1501 together from one or both sides; changing the relative distance of endoscope 2 and distal wall 41; and advancing/retracting the two struts 1501 between portions of scaffold 48 having different intrinsic curvatures (e.g., spring-annealed natural curvatures of struts 1501). Several types of functionally distinguished portions of scaffold 48 are described hereinbelow, and these are optionally coupled to different intrinsic curvatures between each other, and/or within themselves.

In some embodiments, struts 1501 are biased by their sprung (e.g., spring-annealed) shapes to spread out from each other (that is, in opposite lateral directions) when unconfined, e.g., as they leave compound working channel 4. Optionally, they are gathered together again at or after where they contact distal wall 41, e.g., funneled back together by the channels that capture them. Optionally, they return proximally through separate channels. Optionally, struts 1501 only assume the outward bias in sections that exit compound channel 4 after they have been captured, looped, and returned proximal, e.g., via compound channel. The degree of outward bias may be different at different sections, allowing it to be controlled. Optionally, struts 1501 are positioned together with retractor 40 outside the body, and inserted through introducer 1 together with retractor 40. This potentially avoids a need to “capture” their distal ends during remote insertion, and may allow them to spread apart from each other more aggressively. In some embodiments, expansion of struts 15 is performed by use of a separating element, e.g., a wedge-shaped blocker at the distal end of compound working channel 4 that separates them. If separation is forced by an external element, optionally, struts 1501 are spring-biased inward, to help ensure that they meet again for return proximally.

In some embodiments, scaffold 48 comprises a mesh and/or webbing carried between struts 1501 (e.g., struts 1501 configured to spread out along upper loop 48A) and/or extending some amount laterally (circumferentially around a distal-proximal axis of the device) beyond struts 1501.

The mesh and/or webbing has the potential advantage of providing extra support. Struts 1501 optionally comprise a conductive material (e.g., a nitinol alloy). Optionally, struts 1501 are attached on a proximal side to electrical equipment to allow electrical measurements and/or passage of therapeutic (e.g., ablative and/or RF) electrical currents using struts 1501 and/or an electrode portion thereof. In some embodiments, struts 1501 comprise non-conductive material (e.g., a flexible plastic polymer).

In some embodiments, scaffold 48 is configured to perform one or more other functions additionally or alternatively to supporting the working area distal to endoscope 2 to prevent and/or reverse collapse. For example, wire struts 1501 may be configured for cutting (e.g., provided with sharpened edges). Since scaffold 48 can optionally be advanced/retracted independently from either side, scaffold 48 can optionally be configured with a plurality of differently constructed sections along its length; e.g., a section configured for atraumatic support of tissue, a section configured with sharper edges to allow use in cutting, and/or a section with one or more electrodes to allow electrical measurements and/or therapeutic application of electrical energy in one or more locations. In some embodiments, one or more sections of scaffold 48 carry one or more camera detectors and/or light sources (e.g., LEDs), positionable by relative advance/retraction of the two sides of scaffold 48.

Reference is now made to FIGS. 16A-16B, which schematically illustrate endoscopic system configurations comprising introducer 1, endoscope 2, L-shaped retractor 40, deployable scaffold 48 and one or more ultrasound imagers 1600, 1610, according to some embodiments of the present disclosure. Excavated volume 26 (FIG. 16B) within a tissue region (e.g., tumor 31) is held open by retractor 40 and scaffold 48.

In FIG. 16A, ultrasound imager 1600 is shown advanced into a space defined between scaffold 48 and retractor 40 out of a lumen of endoscope 2, e.g., main working channel 3. A planar field of view 1601 is indicated; the planar field of view 1601 can be adjusted by rotating ultrasound imager 1600 around its longitudinal axis.

In FIG. 16B, ultrasound imager 1610 is carried near the distal end of retractor 40. and/or carried by scaffold 48. Image plane 1611 can be oriented as shown, and optionally reoriented by rotation of scaffold 48 and/or retractor 40 relative to tumor 31. Also shown in FIG. 16B is steerable channel 22A, which in this example includes an inner channel tube 1605, extendable from the aperture of curved middle channel tube 21 to reach targets such as target 32.

Ultrasound imagers 1600, 1610 are optionally provided together.

Reference is now made to FIGS. 17A-17B, which schematically illustrates an L-shaped retractor 40 having a distal wall 41 with hole 41d. The figures show an L-shaped retractor 40 wherein the proximal part (e.g., base 42) of the L-shape 41 has a cross section that fits in the lower compound working channel, located between the introducer 1 and the endoscope 2. Angulation of distal wall 41 of the L-shape pushes tissue distal to introducer 1 radially outward as it extends/advances (“downward”, in the orientation of the drawings). Base 42 holds the outward-pushed tissue in place. Hole 41d is formed within distal wall 41 and/or the curvature of region 43. Hole 41d provides potential advantages for enabling visualization of tissue distally beyond the retractor 40, and/or of enabling the passage of tools through it. Camera lens 6 and illuminators 7 are also indicated, along with steerable channel 22A.

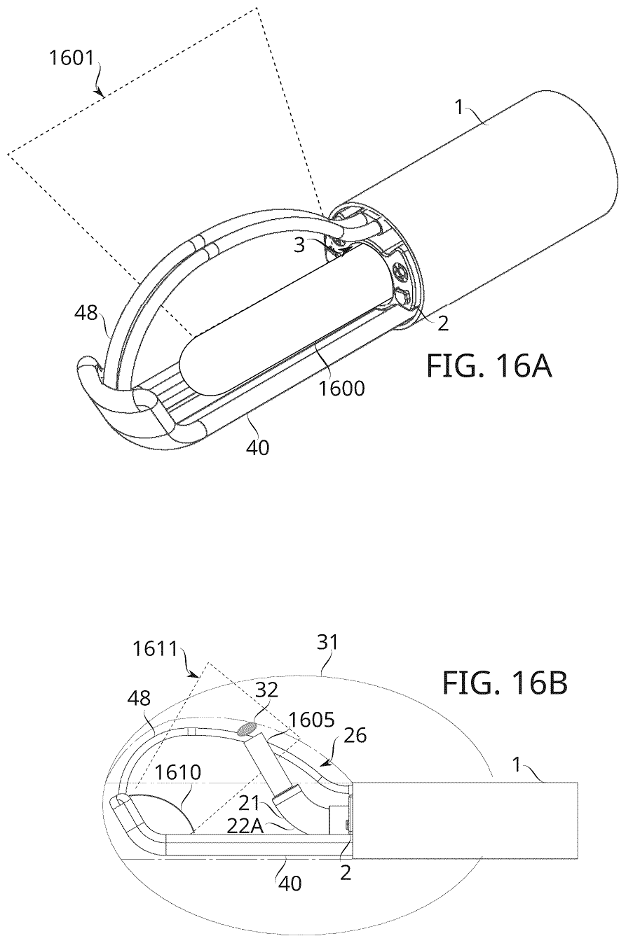

Reference is now made to FIGS. 18A-18C, which schematically illustrate a bendable tool 60, according to some embodiments of the present disclosure. Tool 60 passes distally via a cavity of the introducer/endoscope pair, for example a cavity 46 (e.g., as shown in FIGS. 13D-13E). In some embodiments, tool 60 comprises straight pipe 60a connected to a sprung (e.g., spring-annealed or otherwise predisposed to bend) bendable pipe 60b (e.g., a pipe formed of a superelastic material such as nitinol). Once tool 60 exits the closed cavity 46, sprung bendable pipe 60b curves backward as shown in FIG. 18A. Tool 60 may be moved backward and forward (proximally and distally), for example as shown in FIGS. 18B-18C.

FIG. 18C illustrates an extension from the inner cavity of tool 60 comprising a therapeutic tool 61, optionally used to assist in treating the target 32 from a different direction than access provided to tool 24 via positioning of steerable channel 22A.

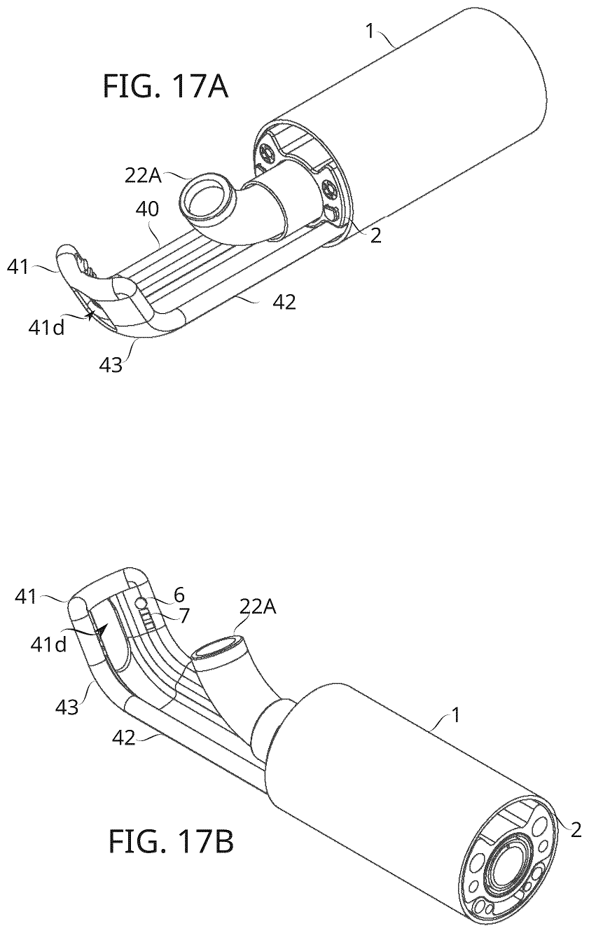

Reference is now made to FIGS. 19A-19C, which schematically represent a steerable bendable tool 60, according to some embodiments of the present disclosure. Tool 60 passes distally via a cavity of the introducer/endoscope pair, for example a cavity 46 as shown in FIGS. 13D-13E. In some embodiments, tool 60 comprises straight pipe 60a, connected to a sprung (e.g., spring-annealed or otherwise predisposed to bend) bendable pipe 60b, and is otherwise generally configured as described in relation to FIGS. 18A-18C. In some embodiments, moreover, bending of a distal portion of tool 60 is adjustable through connection to adjustable cable 60c. Tension of adjustable cable 60c is adjustable, e.g., via a control member leading proximally, not shown. Examples of different angles of bending of tool 60 are illustrated in FIGS. 19B-19C.

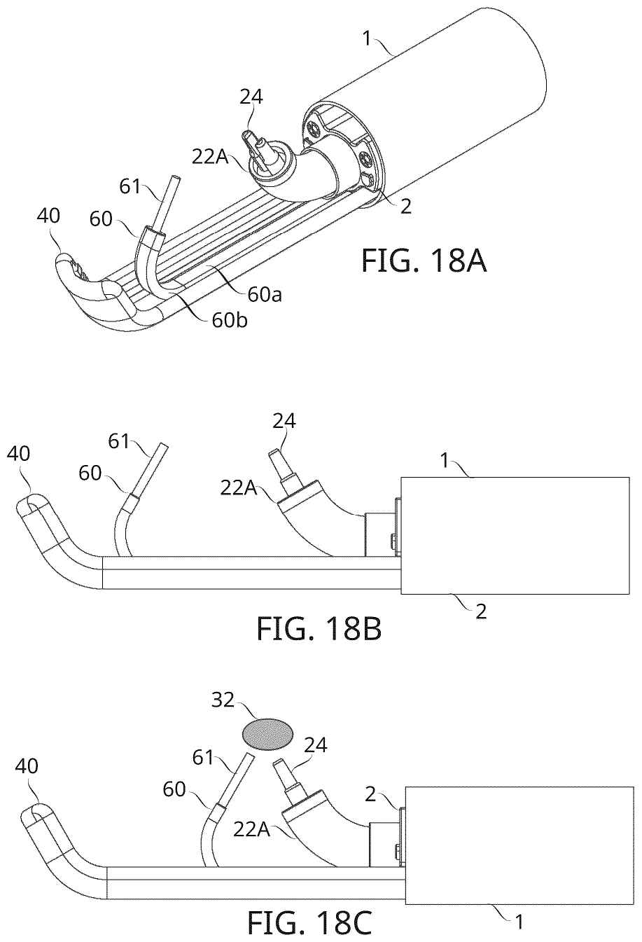

Reference is now made to FIGS. 20A-20C, which schematically represent a configuration of a modular robotic endoscope system 2010, according to some embodiments of the present disclosure. FIG. 20C is a magnified view of a distal region of elements shown in FIG. 20A. The illustrated embodiment of system 2010 is configured to provide separate body-inserted elements comprising endoscope 2001, and steerable working channel 22A. FIG. 20B presents a schematic end-on view of the arrangement of these elements, with endoscope 2001 also labeled CAM (for “camera”), and steerable channel 22A labeled ARM (for “robotic arm”. It should be understood, both generally and particularly with respect to modular aspects of embodiments shown in FIGS. 20A-23C, that embodiments of the present disclosure are not limited to camera and arm configurations shown; they are provided as examples.

Introducer 2000 is preferably straight and stiff, for example as described for introducer 1, although unlike embodiments of introducer 1 illustrated in, e.g., FIG. 1A, introducer 2000 is here shown as non-circular. Introducer 2000 comprises two working channels 2020A and 2020B. These may be (but are not necessarily) identical in size and shape. This can promote flexibility and modularity; e.g., so that the CAM and ARM positions can be swapped and/or duplicated.

Steerable channel 22A may be a channel, for example as described in relation to other figures herein, for example FIGS. 5A-7B and/or 16A-16B. It is shown equipped with bi-polar tool 24, but any other tool may be optionally provided, for example as described in relation to FIGS. 8A-8D. In the example shown, middle channel tube 21 is implemented more particularly as a slotted tube 2021 (FIG. 20C). In this example, at least one spine 2022 of the slotted tube interconnects rings 2023, spaced apart by slots 2024. Slotted tube 2021 is optionally elastically biased (e.g., spring-annealed) to assume a curved shape when unconstrained, while being sufficiently flexible to straighten, e.g., upon withdrawal into outer channel tube 20. Optionally, an additional inner channel tube 1605 is provided, although a tool such as bi-polar tool 24 may be sufficiently self-supporting that inner channel tube 1605 is omitted.

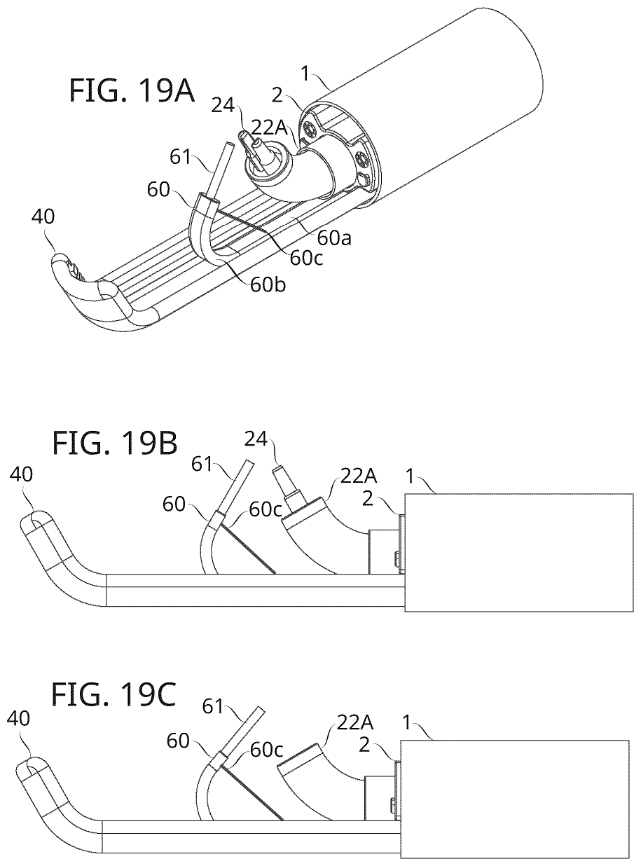

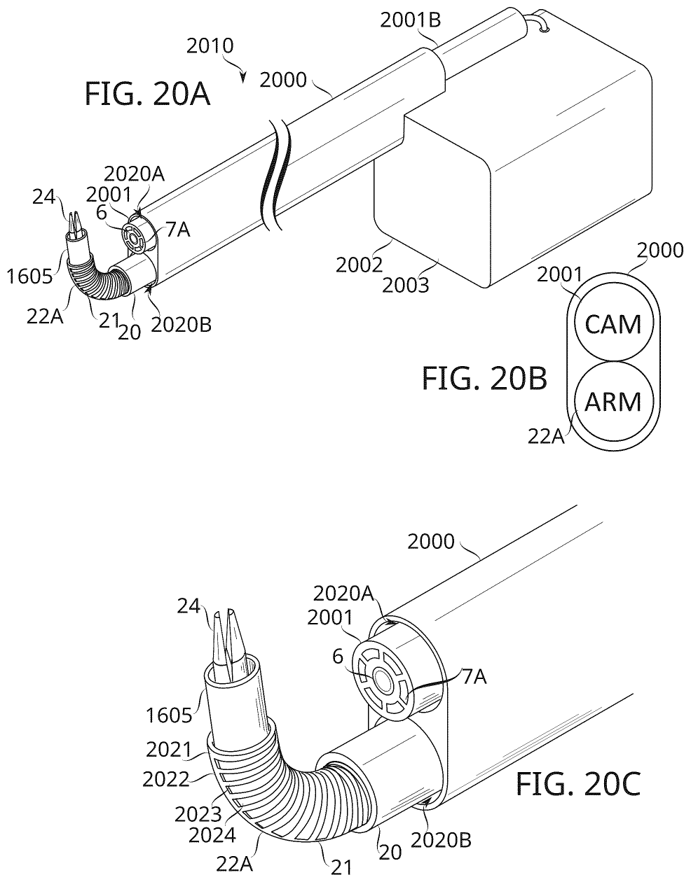

At a proximal side, steerable channel 22A interconnects with robotic controller 2002. Introducer 2000 connects with the enclosure 2003 of robotic controller 2002, which in turn is configured to operate steerable channel 22A. In some embodiments, connection of introducer 2000 and robotic controller 2002 positions proximal-side regions of elements of steerable channel 22A along a side and/or corner of enclosure 2003. Optionally, these elements of steerable channel 22A are positioned with their own cross-sectional areas at least partially, and optionally completely within the proximal-distal axis profile of enclosure 2003 (that is, a profile of enclosure 2003 as seen from a distal-side position). The side and/or corner positioning of introducer 2000 with respect to the enclosure 2003 of robotic controller 2002 potentially allows side-by-side and/or radially arranged configurations using more than one robotic controller 2002, for example as described in relation to FIGS. 21A-23C.

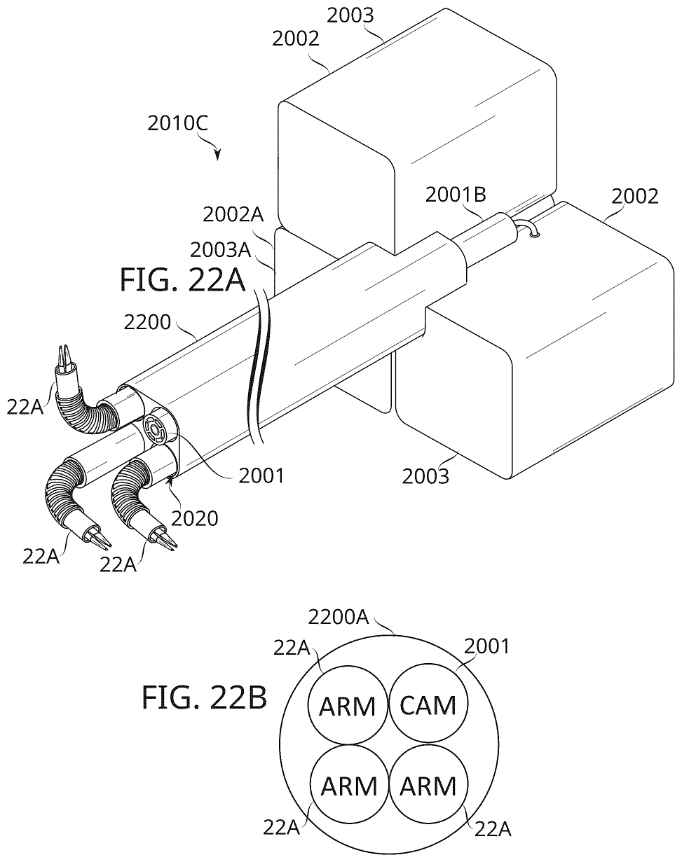

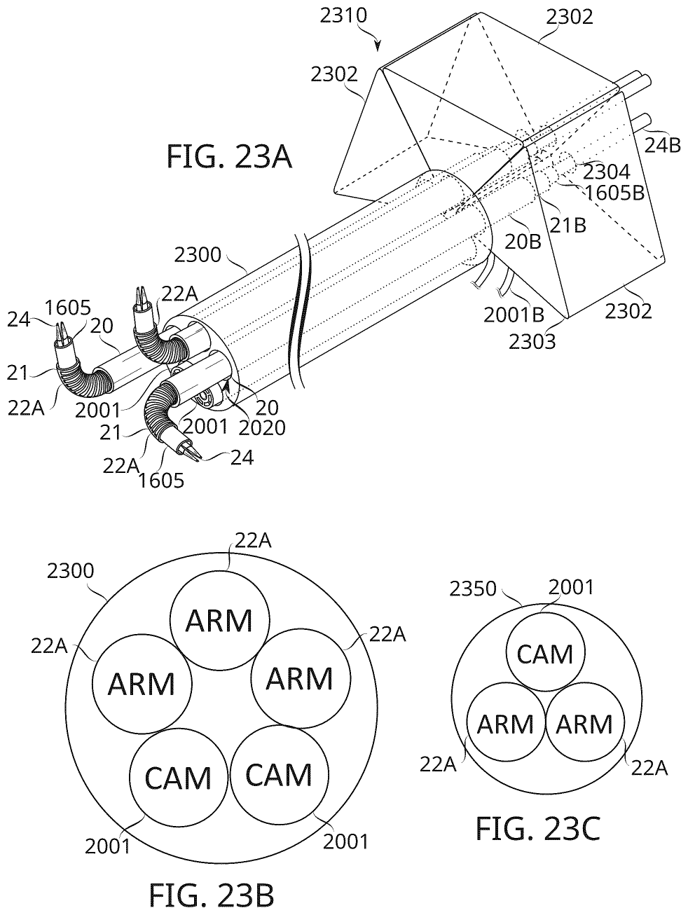

The mechanics of robotic controller 2002 are arranged to engage one or more of the elements of steerable channel 22A, and to actuate their movements (e.g., distally/proximally, and/or rotating). Optionally, robotic controller 2002 also includes actuators for tools, for example, to operate the pincers of bi-polar tool 24. In some embodiments, one or more actuatable elements of steerable channel 22A pass through robotic controller 2002, e.g, to a more proximal module, or to allow direct manual control. Optionally, tool passthrough is provided of actuating element such as cables, wires and/or rods. Apart from its use in actuation, passthrough may be used to provide access to withdraw and insert elements of channel 22A and/or tools used with it, e.g., to exchange elements and/or tools. Passthrough is illustrated, for example, in FIG. 23A.

Endoscope 2001 may comprise any suitable endoscopic capabilities; as shown, it is provided with a camera lens 6 (equipped also with a camera), and illuminator array 7A. Endoscope 2001 can be advanced or retracted through its working channel 2020A by manipulation from a proximal end 2001B. It is shown interconnected with robotic controller 2002 for receiving power/commands for the imaging devices, and/or returning data to robotic controller 2002. These connections are optional, e.g., power may be separately provided, and/or imaging results may be displayed without passing (or at least, not passing directly) into robotic controller 2002. As shown, movements of endoscope 2001 are not themselves robotically controlled, although optionally endoscope 2001 is provided with its own robotic controller, configured for operating its particular degrees of freedom.

Reference is now made to FIGS. 21A-21B, which schematically represent an expanded configuration of a modular robotic endoscope system 2010B, according to some embodiments of the present disclosure. In overview, the elements of system 2010B are the same as for system 2010, except that optionally enclosure 2003A is mirrored with respect to enclosure 2003. To accommodate this change, some portion of the mechanics of robotic controller 2002A are also mirrored. Another option is to design enclosure 2003 so that it can interface equivalently with 30 introducer 2000 in any of at least two orientations which differ from each other by a rotation of 90°. In this case, introducer 2000 may protrude from the “top” of enclosure 2003, or from its “side”, and in the latter case, enclosure 2003 can be rotated to make that side its new top. The arrangement shown allows compact-side-by side positioning of two sets of endoscope and arm, in the same relative orientation e.g., as illustrated in FIG. 21B. The closeness of placement of the robotic arms (e.g., steerable channels 22A) is, for example, limited only by the wall thickness of introducers, while allowing them also to be parallel, e.g., so that they can share a single access way to a target site. However, parallel placement is not required.

In some embodiments, introducers 2000 are used in pairs to simultaneously but separately enter different respective nostrils of a patient, and arranged to converge and/or arrive in parallel at a shared working area within the patient's body, e.g., a target such as a cancer of the pituitary gland, or another brain region accessible by access through the nasal sinuses. The oblong shape of introducer 2000 (in about a 2:1 ratio, e.g., about 10 mm by 5 mm) provides a potential advantage for efficient use of the available cross-sectional area of the nostrils.

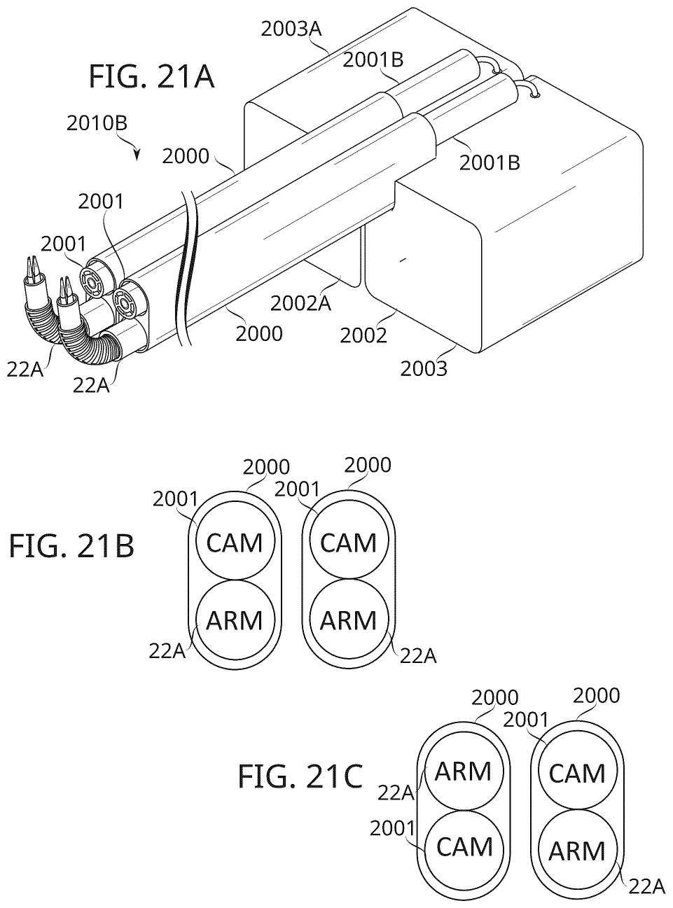

Brief reference is now made to FIG. 21C, which schematically represents an alternative expanded configuration of a modular robotic endoscope system 2010B, according to some embodiments of the present disclosure. In one of the introducers 2000, the positions of ARM and CAM is shown flipped. This allows a second copy of enclosure 2003 to be placed inverted and offset alongside the first enclosure 2003, without a requirement for mirroring its design, or for supporting mating with introducer 2000 in more than one relative orientation. This positioning may be visualized with respect to FIG. 22A, and the two enclosures 2003 positioned at opposite corners of introducer 2200.

It is an aim of some embodiments of the present disclosure to flexibly provide a capability for “manipulator density”; that is, to allow bringing a plurality of stiffly-supported robotic manipulators along parallel routes through a space-constrained passageway. A need for parallel routes may arise in part due to the use of straight and stiff introducers, e.g., for reasons as described in relation to FIGS. 1A-1B.

The enclosure 2003 of robotic controller 2002 is optionally completely self-contained in the role of motion controller. For example, it receives commands in the form of instructions abstracted from hardware specifics, converts these into lower-level commands suitable for components such as motors, and also contains the motors and interfacing hardware (e.g., gears, cables and/or other mechanics which actually contact and move elements such as proximal-side portions of the elements of steerable channel 22A. Sensors (e.g., cameras and/or encoders configured to track and/or verify movement) are optionally provided. Being self-contained may promote modularity and/or simplicity of set-up.

When enclosures are closely arranged, e.g., side-by-side as in FIG. 21A or in another fashion such as is described in relation to FIGS. 22A-23C, there is physically plenty of room in directions radially away from their common center to put all this hardware. Use of this room need not maintain the square aspect ratio shown for enclosures 2003; e.g., the enclosures 2003 can be rectangular, or another shape (for example, FIG. 23A shows a roughly triangular enclosure shape).

However, there may be other constraints on available space for enclosures, e.g., constraints on their weight, or constraints stemming from a need to access the patient in other ways as well.

Accordingly, in some embodiments of the present disclosure, the elements of robotic controller 2002 which are provided within enclosure 2003 may be only a portion of the elements of robotic controller 2002. For example, the contents of enclosure 2003 may implement only what is mechanically needed to move elements, without control logic, and optionally even without motors. Sensor reading and/or external control logic may be implemented, e.g., by a microcontroller or other computing device; communicating as necessary with elements inside enclosure 2003 via a suitable wired or wireless data link. Motor force may be provided from an external motor through a linkage, e.g., a rotating cable. Distributing at least some functions of robotic controller 2002 to enclosures away from enclosure 2003 may assist in achieving a smaller size in locations where space constraints are the most limiting. Although potentially more complex to implement, modularity of design is also possible here, for example by suitable design of the hardware and communication interfaces of enclosure 2003 itself.

Reference is now made to FIGS. 22A-22B, which schematically represent an expanded configuration of a modular robotic endoscope system 2010C, according to some embodiments of the present disclosure.

Again, most components shown are shared with the embodiments of FIGS. 20A-21C. The difference is the use of a single four-position introducer 2200, 2200A. In the sample shown, three arms are provided (e.g., in the form of steerable channel 22A). The remaining port is used for an imaging device, e.g., endoscope 2001. The ports 2020 may all be the same in size and shape, in which case the system may be configured to use any combination of camera devices and arm devices suitable to need (e.g., two of each, or three camera elements and one arm). Optionally, ports 2020 are differentiated, e.g., in embodiments for which endoscope 2001 is differently sized than steerable channels 22A.

Introducer 2200 has a rounded-corner square cross-sectional shape (which may allow a somewhat reduced cross-sectional area for the same port size), while introducer 2200A has a circular cross-sectional shape (which may be preferable, e.g., due to its radial symmetry, which means it cannot be accidentally turned in place to “expand” a tight-fitting body cavity). Optionally, the three robotic controllers 2002, 2002A are the same (and flexible in the relative orientation in which they connect to introducer 2200, 2200A). Optionally, robotic controller 2002A at least partially mirrors the other two (e.g., it has a mirrored enclosure 2003A). The symmetry of introducer 2200 may make special mirroring arrangements unnecessary, however.

Reference is now made to FIGS. 23A-23B, which schematically represent a 5-port modular robotic endoscope system 2310, according to some embodiments of the present disclosure.

In the embodiment of FIGS. 23A-23B, up five ports can be used. System 2310 is adapted to this 5-fold radial symmetry by converting its robotic controllers 2302 to use a roughly triangular-shaped enclosure, e.g., using up to ⅕th of a circular circumference, instead of up to ¼. FIG. 23B illustrates the same population of five ports 2020 as is shown in FIG. 23A—three arms (at top) and two camera elements (bottom). Again, ports 2020 are optionally all identical, but may be different. As for, e.g., FIGS. 21A-22C, other arrangements of port usage are optionally populated according to need.

Brief reference is also made to FIG. 23C, which schematically represents a port arrangement of a 3-port modular robotic endoscope system, according to some embodiments of the present disclosure. In this case, introducer 2350 is sized to provide three ports, populated, for example, with one camera element and two arms as shown. This arrangement allows a single enclosure of a robotic controller to span up to 120° of a circular circumference. Accordingly, it is potentially compatible with (e.g., optionally implemented using) the triangular enclosures 2302 of FIG. 23A, or the square aspect-ratio enclosures of FIGS. 20A, 21A, and 22A.

The remainder of the features now described in relation to FIG. 23A are optionally applied to embodiments of any of FIGS. 20A-22C.

Proximal ends 2001B of endoscopes 2001 are shown disconnected from the robotic controllers 2302, e.g., they may receive power and/or transmit their image signals through a different pathway.

Proximal-side portions of elements of steerable channel 22A are shown as hidden lines within the enclosures 2303 of robotic controllers 2302. It may be noted that proximal side 1605 of inner channel tube 1605 protrudes from proximal side 21B of middle channel tube 21, and this protrudes in turn from proximal side 20B of outer channel tube 20. This exposes access to each of these elements to the internal mechanics (not shown) of the robotic controllers 2302. The depth of enclosure 2302 may be adjusted to suit requirements for longitudinal motion. The depth shown is not to scale with the distal-side positions shown.

Also shown is passthrough port 2304. The proximal side 24B of bi-polar tool 24 is shown passing out of this port, allowing it to be manipulated manually, and/or by an another robotic controller (not shown). Optionally, proximal sides of other elements also protrude through port 2304. This may allow manual override and/or guidance of robotic controller 2302. Additionally or alternatively, robotic controller 2302 may exercise control to guide manual inputs, e.g., based on sensing of mechanical limits, programming that describes the target position, sensing of the tissue environment (e.g., imaged positions of markers), or another source of information.

Additionally or alternatively, using the passthrough port 2304, the functions of robotic controller 2302 may be distributed among a plurality of enclosures positioned along the longitudinal axis of the ports 2020. For example the most distal enclosure may handle outer channel tube 20, the next one (proximally) middle channel tube 21, and the third one inner channel tube 1605. A fourth (or other-numbered) enclosure is optionally responsible for manipulation of tool 24, and optionally reconfigurable or replaceable according to whatever tool is being used.

This approach to robotic control potentially enhances the modularity of systems built according to the descriptions of systems 2010, 2010B, and/or 2010C. In some embodiments, if one of the tubes, e.g., inner channel tube 1605, is unneeded for a particular port configuration, its corresponding enclosure is optionally omitted. Optionally, if a different design, e.g., of a middle channel tube 21 is needed (e.g., one with a different radius of curvature upon release), its own specialized controller enclosure is optionally swapped in. Controller enclosures optionally are capable of driving a plurality of different elements. They may sense which channel tube type and/or channel tube variant they are installed with (e.g., via RFID chip, contact pin sensing, or another method), and adjust their operation accordingly, if possible. Otherwise, they may report their incompatibility to operate with the current configuration.

It should be understood that any of the features described herein relating to robotic control of embodiments of FIGS. 20A-23C, are optionally provided for embodiments described in relation to FIGS. 1A-19C, insofar as they are compatible. For example, robotic control feature which are applicable to a single steerable working channel 22A and/or a tool positioned therein, are optionally provided to any embodiment making use of such as steerable working channel 22A and/or such a tool, even if described in the context of a different introducer and/or endoscope.

### General

It is expected that during the life of a patent maturing from this application many relevant robotic surgical tools will be developed; the scope of the term robotic surgical tool is intended to include all such new technologies a priori.

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, to the same extent as if each individual publication, patent or patent application was specifically and individually noted when referenced that it is to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

In the claims:

1. An endoscopic guide for brain surgery, comprising:
(a) a body having a body lumen with a distal side and a proximal side;
(b) a tool lumen defined within said body lumen and sized for transfer of a brain surgery tool therethrough to a distal opening on the distal side of the body lumen; and
(c) at least two distal-facing imagers positioned with respective fields of view each including, from a different respective circumferential position around the distal opening, a region surrounding an axis extending longitudinally along the tool lumen out of the distal opening;
wherein the distal opening subtends at least 70° of a total circumference surrounding each of the two distal-facing imagers.

2-3. (canceled)

4. The endoscopic guide according to claim 1, wherein a distance between each of said at least two imagers and said tool lumen is less than 3 mm.

5. The endoscopic guide according to claim 1, comprising at least one additional channel within said body lumen and outside of said tool lumen.

6. The endoscopic guide according claim 5, wherein said at least one additional channel and said tool lumen and said at least two imagers rotate separately from said body lumen.

7. The endoscopic guide according to claim 5, wherein said at one additional channel and said tool lumen and said at least two imagers rotate upon rotation of said body.

8. The endoscopic guide according to claim 1, further comprising a said brain surgery tool, said brain surgery tool comprising one or more of suction, electrical cauterization and tissue cutting tools;
wherein said brain surgery tool rotates within the tool lumen, and comprises a bending region through which it bends; and extends longitudinally along a longitudinal axis of a portion of the brain surgery tool positioned distally beyond the bending region.

9. (canceled)

10. The endoscopic guide according to claim 8, wherein said brain surgery tool retracts proximally along a path including a path portion along the longitudinal axis of the portion of the brain surgery tool positioned distally beyond the bending region, and a path portion passing through the bent bending region.

11. The endoscopic guide according to claim 8, wherein said guide or circuitry attached thereto generates an indication when a tip of said tool extends outside of a predefined region distal to and extending from the distal side of said body lumen.

12-18. (canceled)

19. The endoscopic guide according to claim 1, comprising a tissue support, sized and shaped to reduce ingress of surrounding tissue into a body volume within a region distal to and extending from the distal side of said body lumen.

20. (canceled)

21. The endoscopic guide according to claim 19, comprising an ultrasound transducer positioned on a distal portion of the tissue support, and oriented to image in a proximal direction from its position.

22-28. (canceled)

29. The endoscopic guide according to claim 19, wherein said tissue support is sized to insert through an auxiliary channel within said body lumen.

30-34. (canceled)

35. The endoscopic guide according to claim 19, wherein said tissue support defines at least one suction aperture facing away from said body volume.

36-38. (canceled)

39. The endoscopic guide according to claim 19, wherein said tissue support is positionable to prevent contact of said brain surgery tool with sensitive tissue outside of said body volume.

40. The endoscopic guide according to claim 1, comprising at least one narrow tissue supporter extending distally from said body lumen.

41. (canceled)

42. The endoscopic guide according to claim 40, wherein said at least one narrow tissue supporter comprises two separate wires or is in the form of a loop, with each of two sides of the loop extending distally out of the body lumen.

43-46. (canceled)

47. The endoscopic guide according to claim 40, wherein said at least one narrow tissue supporter is flexible enough to move out of the way when contacted by said surgical tool.

48. The endoscopic guide according to claim 40, wherein said at least one narrow tissue supporter is re-positionable to mark a tumor or other tissue to be removed or to mark a tissue to be avoided, while the distal opening remains in place.

49. The endoscopic guide according to claim 40, comprising a tissue support having a base extending axially along a surface of body volume distal to and extending from the distal side of said body lumen;
wherein said at least one narrow tissue supporter lies on an opposite side of said body volume from said base and extends to rest against said base;
wherein said tissue support restricts axial movement of said at least one narrow tissue supporter and thereby converts axial movement thereof into lateral and/or circumferential movement thereof.

50-70. (canceled)
